(19) United States
(12) Patent Application Publication
(10) Pub. No.: US 2026/0193231 A1
(43) Pub. Date: Jul. 9, 2026

(54) QUATERNARY AMMONIUM CYANINE DYES

(71) Applicants: GEORGIA STATE UNIVERSITY RESEARCH FOUNDATION, INC., Atlanta, GA (US); The Board of Regents of the University of Oklahoma, Norman, OK (US)

(72) Inventors: Maged HENARY, Lawrenceville, GA (US); Emmanuel Ramsey BUABENG, Atlanta, GA (US); Lacey R. MCNALLY, Edmond, OK (US)

(21) Appl. No.: 18/869,566

(22) PCT Filed: Jun. 1, 2023
(86) PCT No.: PCT/US2023/024093

§ 371 Date: Nov. 26, 2024

Related U.S. Application Data
(60) Provisional application No. 63/347,785, filed on Jun. 1, 2022.

(51) Int. Cl. C07D 401/14; C09B 23/14

(52) U.S. Cl. CPC C07D 401/14; C09B 23/145

(57) ABSTRACT

Disclosed herein are quaternary ammonium cyanine dyes. The dyes exhibit different absorption/emission profiles at different pH levels. The compounds can be used to detect Lewis acidic species, including metal ions and protons, for instance as pH probes. The compounds can be used as agents for biological assays, including diagnostics like optical, fluorescent, and optoacoustic imaging, especially in the detection of cancerous and other unhealthy cells.

### CROSS-REFERENCE TO RELATED APPLICATION

This application claims the benefit of U.S. Provisional Application 63/347,785, filed Jun. 1, 2022, the contents of which are hereby incorporated in their entirety.

### STATEMENT OF GOVERNMENT SUPPORT

This invention was made with government support under grant/contract number R01CA205941, awarded by the National Institutes of Health. The government has certain rights in the invention.

### FIELD OF THE INVENTION

Disclosed herein are quaternary ammonium cyanine dyes. The dyes exhibit different absorption/emission profiles at different pH levels. In some embodiments, the quaternary ammonium cyanine dyes can be used to detect Lewis acidic species, including metal ions and protons. In some embodiments, the quaternary ammonium cyanine dyes can be used as pH probes. In some embodiments, the quaternary ammonium cyanine dyes can be used as agents for biological assays, including diagnostics like optical, fluorescent, and optoacoustic imaging. In some embodiments, the quaternary ammonium cyanine dyes can be used in optoacoustic imaging of cancerous cells.

### BACKGROUND

Photomedicine broadly refers to the use of light for diagnostic or therapeutic procedures, including optical imaging and photoacoustic imaging, photothermal therapy (thermal ablation of cells) and photodynamic therapy (reactive oxygen species induced apoptosis or necrosis). The low toxicity of light coupled with the direct control of localization and dosage make phototherapy a promising avenue for increasing the therapeutic index of disease treatment. Near-infrared (700-2,000 nm) radiation is especially attractive as these wavelengths can traverse increasingly large distances through tissue. Since water is transparent at these wavelengths, there are less noise and confounding signals as well. Near-infrared may be subdivided between NIR-I (700-1,000 nm) and NIR-II (1,000-2,000 nm). NIR I and NIR II materials are desirable for tissue imaging due to the deeper penetration of light, minimal tissue damage, and high spatial resolution as a result of low autofluorescence in the NIR I and NIR II regions. Several examples of known NIR I dyes are derived from common fluorescent dye scaffolds such as cyanine, phthalocyanine and porphyrin, squaraine, BODIPY analogs, benzo[c]heterocycle, and xanthene derivatives.

Photoacoustic Imaging (PAI) is an emerging medical imaging modality that is based on the phenomenon of conversion of optical energy into acoustic energy. PAI offers distinct advantages over other strictly optical imaging methods such as fluorescence because physiological tissue poses considerably less interference for acoustic waves than it does for light.

When performing cancer surgery with intent of radically remove cancer and metastases, distinguishing healthy from cancerous tissue can be difficult, which can lead to either removal of excess healthy tissue or incomplete removal of cancerous tissue, increasing the likelihood of recurrence and/or additional surgeries. Fluorescence-based imaging has been explored to distinguish healthy and cancerous cells. Various agents can be conjugated to antibodies and other targeting moieties. However, antibody imaging probes share common disadvantages as compared to small organic fluorophores, such as their relatively long retention times in non-targeted tissues, slow clearance from circulation, and extensive condition optimization requirements. Furthermore, due to poor cell-membrane permeability, antibody-based imaging is limited in its applications to cell-surface biomarkers.

Certain pathophysiological states are characterized by abnormal cellular pH levels relative to healthy cells. Cells experience increased metabolic processes (as often found in cancerous cells) can in certain cases be observed to have lower pH than non-cancerous cells. Some pathophysiologic states are characterized by different concentrations of certain metal ions.

There remains a need for imaging agents and methods that accurately detect tissue systems characterized by abnormal pH levels and/or the presence or absence of various metal ions. There remains a need for improved methods of diagnosing diseases and disorders characterized by abnormal pH and/or metal concentrations. There remains a need for detecting cancerous cells in early stages, before solid tumors have developed. Some cancers, for instance pancreatic cancers, are often not detected until the late stages of disease, when treatment is difficult or impossible. In contrast, early stage detection and intervention can greatly improve quality of life and survival. There remains a need for improved methods of differentiate healthy and non-healthy, e.g., cancerous, tissues during a real-time surgical procedure. This would allow more precise removal of cancerous and/or diseased tissue from locations within, surrounding, and/or adjacent to critical organs or tissue, especially where significant harm may result from damage to or removal of healthy tissue. This would also reduce the amount of healthy tissue that is removed, and would reduce the risk of recurrence.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts the influence of protonation over the absorbance spectra of 52. Conc. (10 μM). pH range (1-10). A gradual bathochromic shift in the absorbance wavelength from 552 nm to 703 nm.

FIG. 2 depicts the responsiveness of compound 52 varying the pH of the buffer solution (PBS). Conc. of compound 52, 1.3 μM. A. Excitation wavelength (550 nm). B. Excitation wavelength (703 nm).

FIG. 3 depicts the influence of protonation over the absorbance spectra of compound 53 Conc. (10 μM). pH range (1-10). A gradual bathochromic shift in the absorbance wavelength from 552 nm to 703 nm. B. N-methyl Piperazine moiety adopt a chair conformation in the ground state (neutral form) upon protonation it changes conformation to allow for protonation of the other nitrogen atom.

FIG. 4 depicts the responsiveness of compound 53 with varying pH (1-10) of the buffer solution (PBS) Conc. of dye (1.3 μM) and excitation wavelength (550 nm). B. Excitation wavelength (703 nm).

FIG. 5 depicts the influence of protonation over the A) absorbance b) emission spectra of compound 54 Conc. (10 μM). pH range (1-10).

FIG. 6 depicts the responsiveness of compound 54 with varying pH of the buffer solution (PBS) used. Conc. of dye (1.3 μM). A) Fluorescence spectra obtained at the excitation wavelength of 703 nm). B) pKa was obtained after from the sigmoidal Hill plot.

FIG. 7 depicts the influence of protonation over the A) absorbance b) emission spectra of compound 55 Conc. (10 μM). pH range (1-10). A gradual bathochromic shift in the absorbance wavelength from 552 nm to 703 nm and systematic increase in the fluorescence intensity. Excitation wavelength (552 nm).

FIG. 8 depicts the responsiveness of compound 55 with varying pH of the buffer solution (PBS) used. Conc. of dye (1.3 μM). A) Fluorescence spectra obtained at the excitation wavelength of 703 nm). B) pKa was obtained after from the sigmoidal Hill plot.

FIG. 9 depicts the influence of protonation over the A) absorbance b) emission spectra of compound 56 Conc. (10 μM). pH range (1-10). A gradual bathochromic shift in the absorbance wavelength from 552 nm to 703 nm and systematic increase in the fluorescence intensity. Excitation wavelength (552 nm).

FIG. 10 depicts the responsiveness of compound 56 with varying pH of the buffer solution (PBS) used. Conc. of dye (1.3 μM). A) Fluorescence spectra obtained at the excitation wavelength of 703 nm). B) pKa was obtained after from the sigmoidal Hill plot.

FIG. 11 depicts the influence of protonation over the A) absorbance b) emission spectra of compound 57 Conc. (10 μM). pH range (1-10). A gradual bathochromic shift in the absorbance wavelength from 552 nm to 703 nm and systematic increase in the fluorescence intensity. Excitation wavelength (552 nm).

FIG. 12 depicts the responsiveness of compound 57 with varying pH of the buffer solution (PBS) used. Conc. of dye (1.3 μM). A) Fluorescence spectra obtained at the excitation wavelength of 703 nm). B) pKa was obtained after from the sigmoidal Hill plot.

FIG. 13 depicts a plot of calculated pka for synthesized aminocyanine dyes 52-57 obtained after employing the DoseRep function to fit the fluorescence or absorbance data.

FIG. 14 depicts photostability studies of compound 52-57 under continuous irradiation with Xenon lamp 150 W for 2 h. The rate of photobleaching of the compounds 52-57 were determined based on the reduced fluorescence intensity upon continuous irradiating with light (excitation wavelength, 703 nm for compounds 52-57 and 800 nm for ICG). Commercially available dye, ICG was used as a reference.

FIG. 15 depicts Multispectral optoacoustic tomography (MSOT) signal strength/dye concentration for compound 52 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 16 depicts Multispectral optoacoustic tomography (MSOT) signal strength/dye concentration for compound 52 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals FIG. 17 depicts multispectral optoacoustic tomography (MSOT) signal strength/dye concentration for compound 53 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 18 depicts multispectral optoacoustic tomography (MSOT) signal strength/dye concentration for compound 53 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 19 depicts multispectral optoacoustic tomography (MSOT) signal strength/dye concentration for compound 54 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 20 depicts multispectral optoacoustic tomography (MSOT) signal strength/dye concentration for compound 54 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 21 depicts multispectral optoacoustic tomography (MSOT) signal strength/dye concentration for compound 55 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 22 depicts multispectral optoacoustic tomography (MSOT) signal strength/dye concentration for compound 55 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 23 depicts multispectral optoacoustic tomography (MSOT) signal strength/dye concentration for compound 56 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 24 depicts multispectral optoacoustic tomography (MSOT) signal strength/dye concentration for compound 56 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 25 depicts multispectral optoacoustic tomography (MSOT) signal strength/dye concentration for compound 57 at pH=7.4 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 26 depicts multispectral optoacoustic tomography (MSOT) signal strength/dye concentration for compound 57 at pH=6.6 and its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 27 depicts a summary of optoacoustic signal intensities for compounds 52-57. At 1 mg/mL, each dye was evaluated in the tissue phantom using the MSOT system. The phantoms were scanned at 680 nm through 900 nm at 50 nm wavelength intervals.

FIG. 28 depicts a summary of optoacoustic signal intensities for compounds 58-63. At 1 mg/mL, each dye was evaluated in the tissue phantom using the MSOT system. The phantoms were scanned at 680 nm through 900 nm at 50 nm wavelength intervals.

FIG. 29 depicts a summary of optoacoustic signal intensities for compounds 64-69. At 1 mg/mL, each dye was evaluated in the tissue phantom using the MSOT system. The phantoms were scanned at 680 nm through 900 nm at 50 nm wavelength intervals.

FIG. 30 depicts the chemical structure of compound 86 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 31 depicts the chemical structure of compound 87 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 32 depicts the chemical structure of compound 88 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 33 depicts the chemical structure of compound 89 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 34 depicts the chemical structure of compound 90 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 35 depicts the chemical structure of compound 91 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 36 depicts the chemical structure of compound 92 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 37 depicts the chemical structure of compound 93 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 38 depicts the chemical structure of compound 94 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 39 depicts the chemical structure of compound 95 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 40 depicts the chemical structure of compound 96 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 41 depicts the chemical structure of compound 97 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 42 depicts the chemical structure of compound 98 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 43 depicts the chemical structure of compound 99 and its multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 44 depicts the chemical structure of compounds 54, 100 & 101 and multispectral optoacoustic tomography (MSOT) signal strength/dye concentration at pH=7.4 and 6.6 as well as its corresponding optoacoustic image generated. At 1 mg/mL, the dye was evaluated in the tissue phantom using the MSOT system. The phantoms were scanned at 680 nm through 900 nm at 50 nm wavelength intervals.

FIG. 45 depicts tissue pH. Tissue pH of mouse organs was assessed in 10 mice. Pancreatic tumors were significantly more acidic than liver, kidney, or fibrous tissue p=0.02, 0.04, 0.03. pH is log scale.

FIG. 46 depicts human PDAC tumor pH measured with a microsensor. PDAC tumor (20 cases) was more acidic than non-involved pancreas tissue (14 cases). Tumor pH ranged from 6.29-6.61 vs non-malignant pH of 7.36-7.68. *p=0.002. pH is log scale.

FIG. 47 depicts optoacoustic imaging of compound 101 at differing pHs in tissue mimicking phantoms. Compound 101 was assessed with MSOT to identify a spectral change between pH 7.4 and 6.8-6.0. The ability to generate a completely differing optoacoustic spectra solely based on a change in pH is truly unique. The reproducibility of the acidic pH compound 101 spectra between 6.8-6.0 specifically is optimal for identifying potential pH within tumor heterogeneity.

FIG. 48 depicts toxicity testing results for compound 101.

FIG. 49 depicts light diffusing tissue mimicking phantoms were constructed of standard materials. Compound 101 at 200 μM were inserted into the wells of the phantom and imaged using MSOT. Optoacoustic signals were spectrally unmixed based upon the spectrum of each individual dye.

FIG. 50 depicts optoacoustic signal intensity of commercially available Methylene blue and ICG with compounds 54, 95, and 101 at 200 uM. (A) Commercially available Methylene blue and ICG OA signal compared to compound 95. (B) Compound 54 generated >1000% increase from ICG. (*P<0.05, **P<0.01).

FIG. 51 depicts optoacoustic signal intensity for compounds 95, 54, 101, and ICG in tissue mimicking phantoms. (A) Light diffusing tissue mimicking phantoms were constructed of standard materials. Compounds at 200 μM were inserted into the wells of the phantom and imaged using MSOT. Optoacoustic signals were spectrally unmixed based upon the spectrum of each individual dye.

FIG. 52 depicts toxicity studies of compounds 54 and 101 compared to ICG.

FIG. 53 depicts imaging of mouse injected with 0.1 g/kg of compound 101. Mice were imaged 10 min following iv injection using standard MSOT procedures. Compound 101 Acid spectrum was used to identify positive tumor signal. T=PDAC tumor; St=Stomach; K=Kidney; Sp=Spine.

FIG. 54 depicts tumor and liver removed from mouse in FIG. 130, placed into tissue phantoms, and imaged with MSOT. (A) Compound 101 Acid spectrum was used to identify positive tumor signal and liver. PDAC had significant compound 101 Acid signal. Uninvolved pancreas had minor signals, which could be due to disseminated disease. Liver and kidney were negative for compound 101 acid spectrum.

FIG. 55A depicts UV-Vis absorption spectra of compound 55 in water (5.5 uM of dye with 2.2 mM of cations) FIG. 55B UV-Vis absorption spectrum of compound 55 (Conc. 5.5 uM) with increasing Cu²⁺ ion concentration (0 to 30 mM) in water FIG. 56 depicts a fluorescence spectrum of compound 55 (dye concentration=0.9 uM) upon Cu²⁺ titration (0 to 0.9 mM) at wavelength of excitation at FIG. 56A is 590 nm; FIG. 56B is 815 nm.

FIG. 57 depicts competitive studies of compound 55 (Conc. 5.5 uM) and with all cations 10 mM in water.

FIG. 58 depicts time dependent studies of compound 55 (Conc 5.5 uM) with 10 mM Cu²⁺ ion in water.

FIG. 59A depicts UV-Vis absorption spectra of compound 56 in methanol (5.5 uM of dye with 2.2 mM of cations). FIG. 59B depicts UV-Vis absorption spectrum of compound 56 (Conc. 5.5 uM) with increasing Cu²⁺ ion concentration (0 to 4.5 mM) in methanol.

FIG. 60 depict fluorescence spectrum of compound 56 (dye concentration=0.9 uM) upon Cu²⁺ titration (0 to 9.09 mM) at wavelength of excitation at 550 nm (FIG. 60A) and 859 nm (FIG. 60B).

FIG. 61 depicts competitive studies of compound 56 (Conc. 5.5 uM) and with all cations 10 mM in methanol.

FIG. 62 depicts time dependent studies of compound 56 (Conc 5.5 uM) with 10 mM Cu²⁺ ion in methanol.

FIG. 63A depicts UV-Vis absorption spectra of compound 56 in (5.5 uM of dye with 2.2 mM of cations). FIG. 63B depicts UV-Vis absorption spectrum of compound 56 (Conc. 5.5 uM) with increasing Cu²⁺ ion concentration (2.27 to 22.72 mM) in H₂O:EtOH (1:1) mixture.

FIG. 64 depicts fluorescence spectrum of compound 56 (dye concentration=0.075 uM) upon Cu²⁺ titration (0 to 0.45 mM) at excitation wavelength of 550 nm (FIG. 64A) and 800 nm (FIG. 64B).

FIG. 65 depicts competitive studies of compound 56 (Conc. 5.5 uM) and with all cations 10 mM in H₂O:EtOH (1:1) mixture.

FIG. 66 depicts competitive studies of compound 56 (Conc. 5.5 uM) and with all cations 10 mM in H₂O:EtOH (1:1) mixture.

FIG. 67 depicts absorption spectra of compound 97 (10 uM) in methonal upon titration with (FIG. 67A) 0 to 2.0 mM of Al³⁺; (FIG. 67B) 0 to 2.0 mM of Cr³⁺; (FIG. 67C) 0 to 2.0 mM of Fe³⁺; and (FIG. 67D) 0.45 mM other ions FIG. 68A depicts absorbance spectrum of probe 97 (10 uM) in methanol upon titration with some selected biologically relevant metal ions (0.45 mM). FIG. 68B depicts colorimetric response of probe 97 with various metal ions (0.45 mM) examined.

FIG. 69 depicts absorbance spectrum of probe 97 (10 uM) in H₂O:EtOH (3:7) upon titration with (FIG. 69A) some selected biologically relevant metal ions excluding Fe³⁺ ion (0.45 mM); (FIG. 69B) all ions; (FIG. 69C) increasing amount of Fe³⁺ ion (0.023 mM to 0.68 mM); and (FIG. 69D) emission spectrum of probe 97 with increasing amount of Fe³⁺ ion (0.0 μM to 0.45 μM) at excitation wavelength of 596 nm.

FIG. 70A depicts fluorimetric response of probe 97 upon titration with increasing amount of Fe³⁺ ion (0.09 M to 0.45 μM) at excitation wavelength of 615 nm. FIG. 70B depicts colorimetric response of probe 97 (10 μM) with various metal ions (0.45 mM) examined in H₂O:EtOH (3:7) mixture.

FIG. 71 depicts competitive studies of compound 97 (Conc. 10.0 μM) and with all cations 0.45 mM in H₂O:EtOH (3:7) mixture.

FIG. 72 depicts time dependent studies of compound 97 (Conc. 10.0 μM) with Fe³⁺ ion 0.45 mM in H₂O:EtOH (3:7) mixture

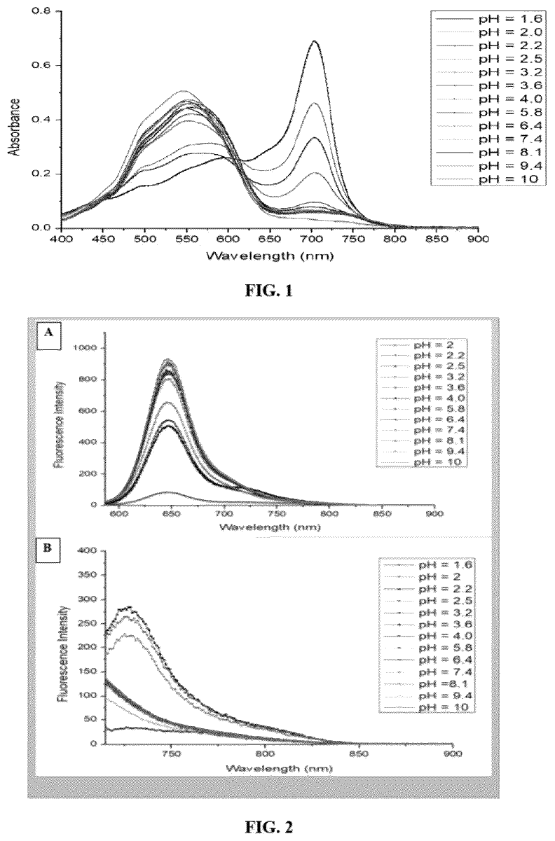

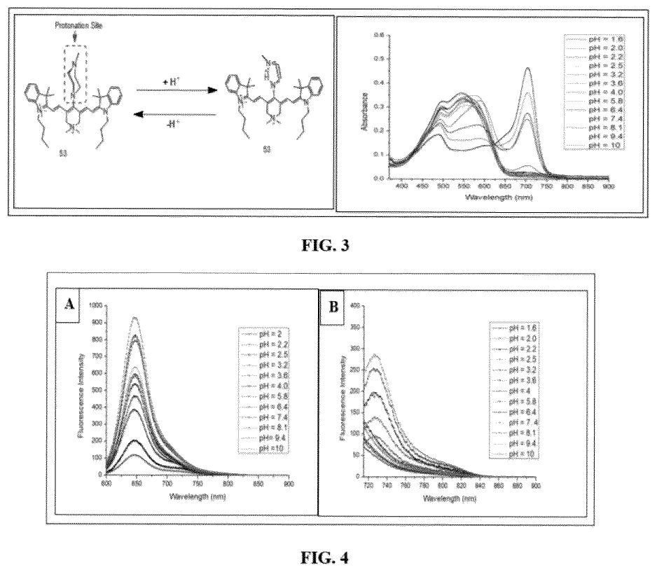

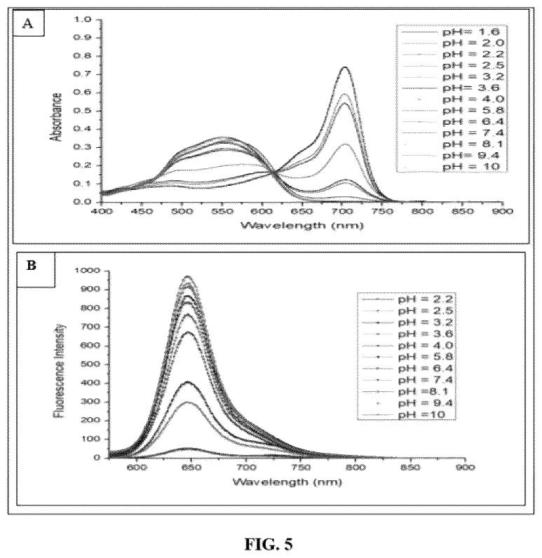

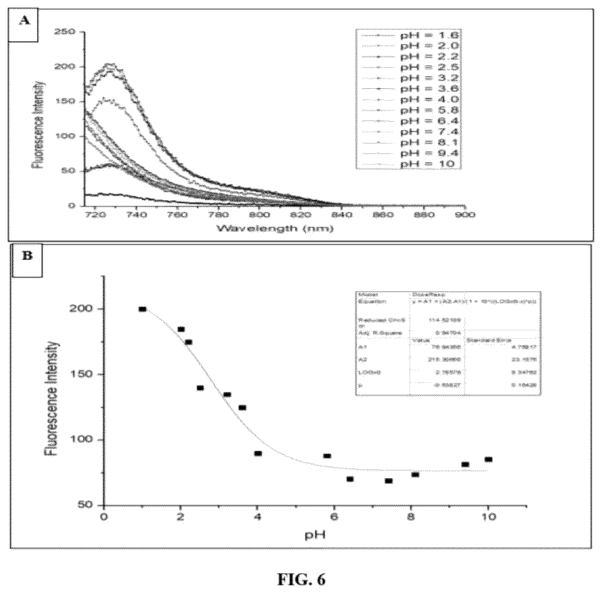

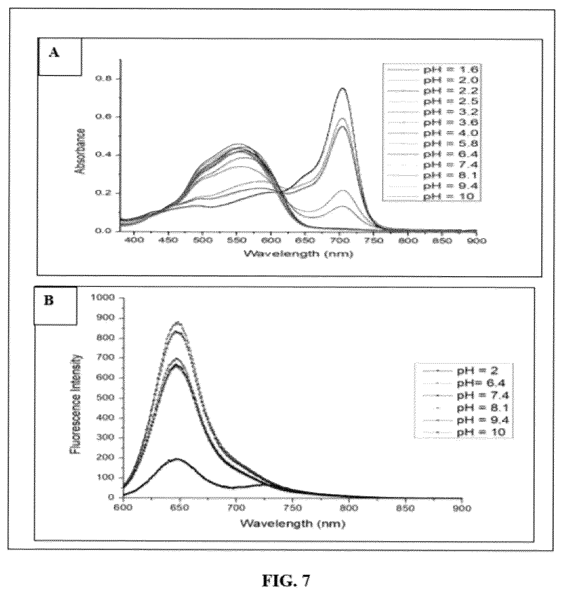

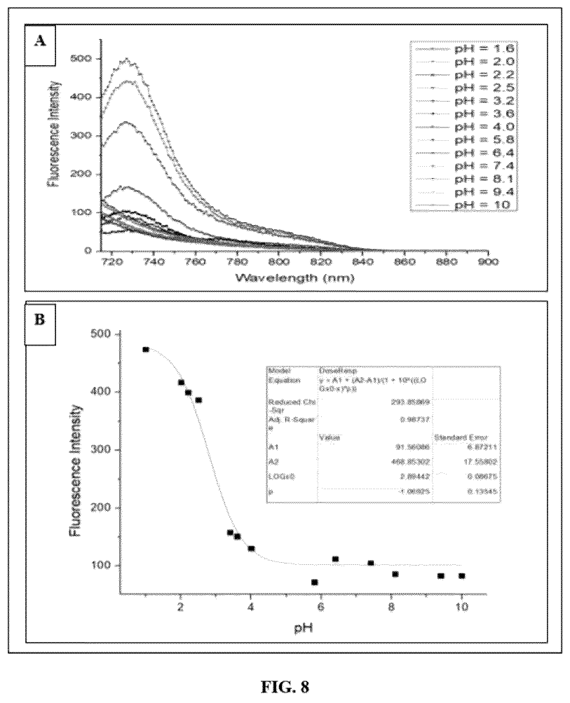

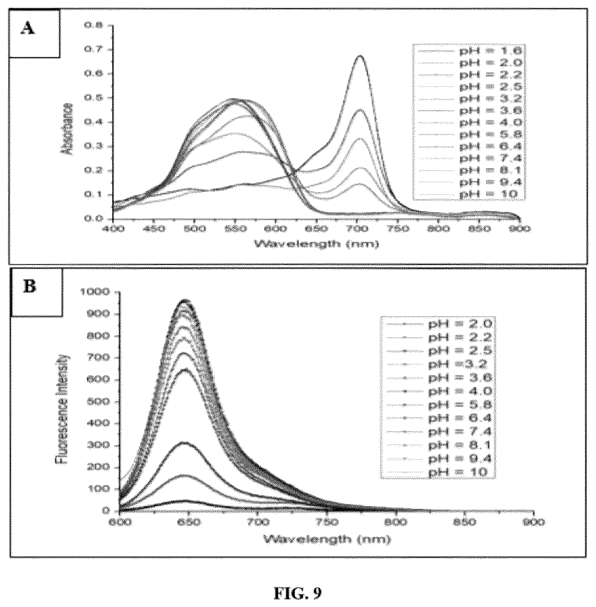

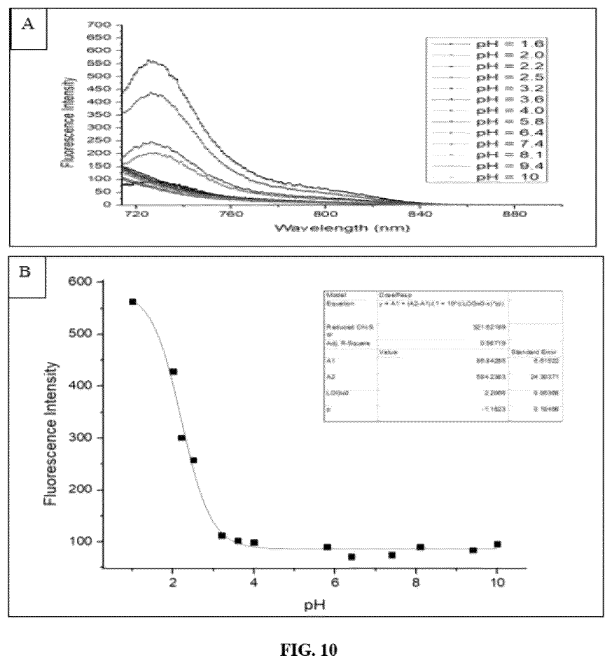

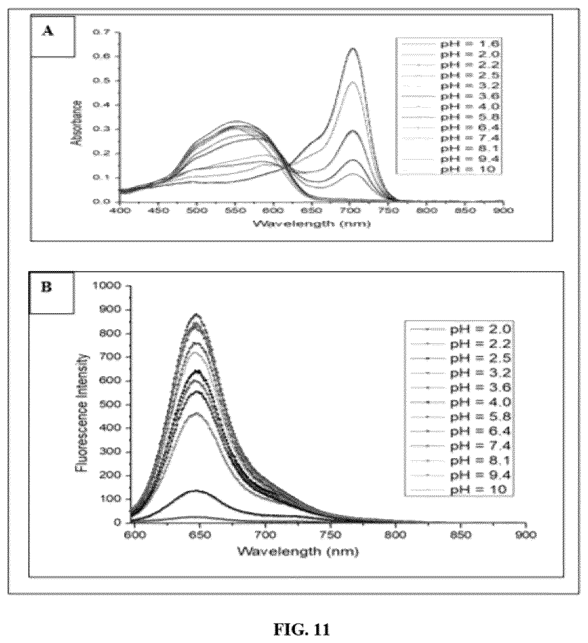

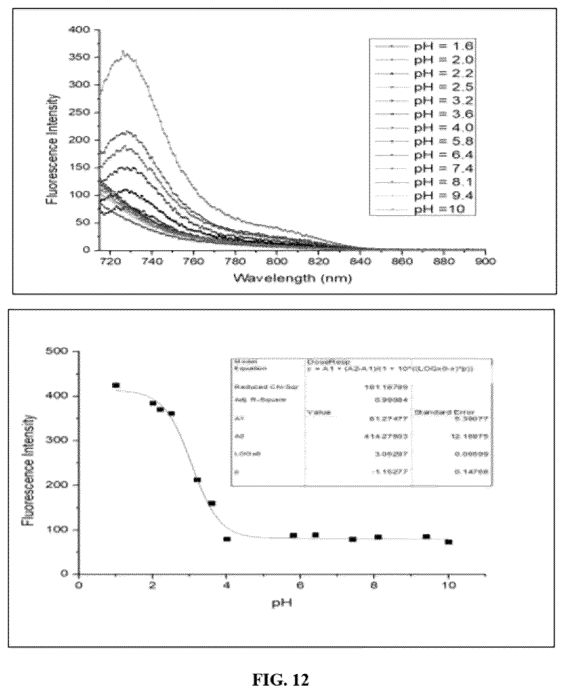

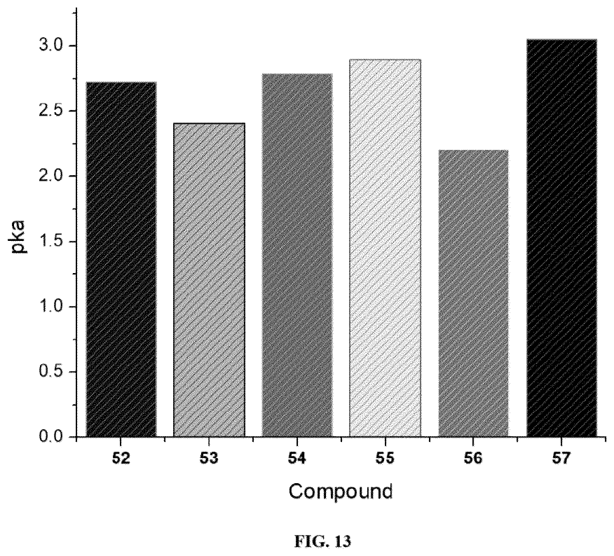

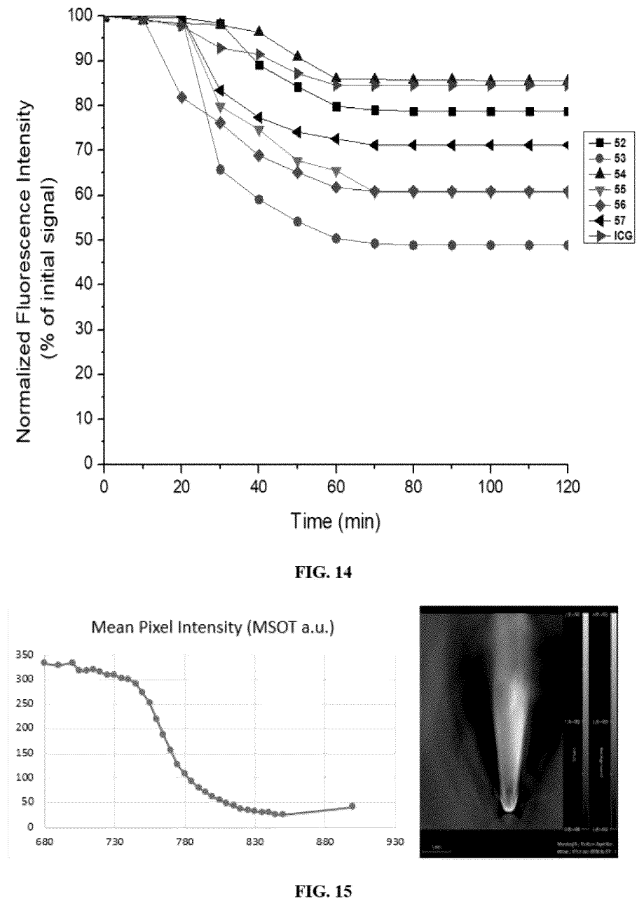

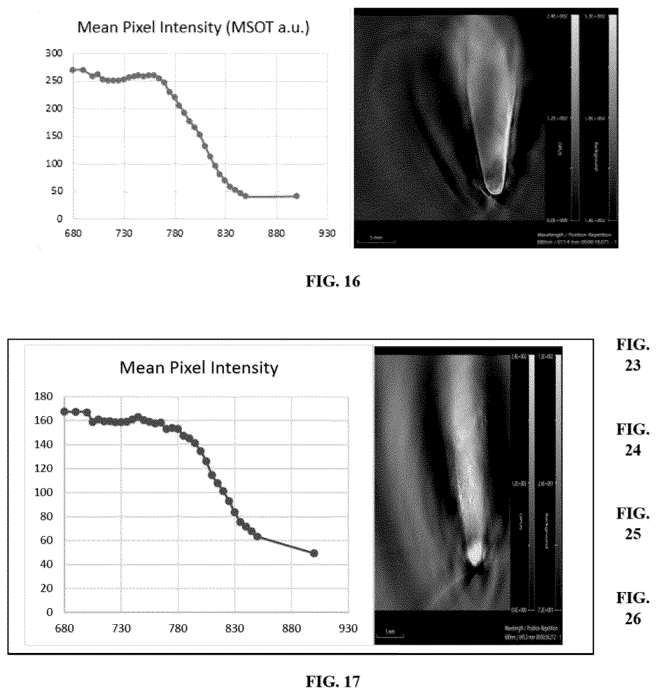

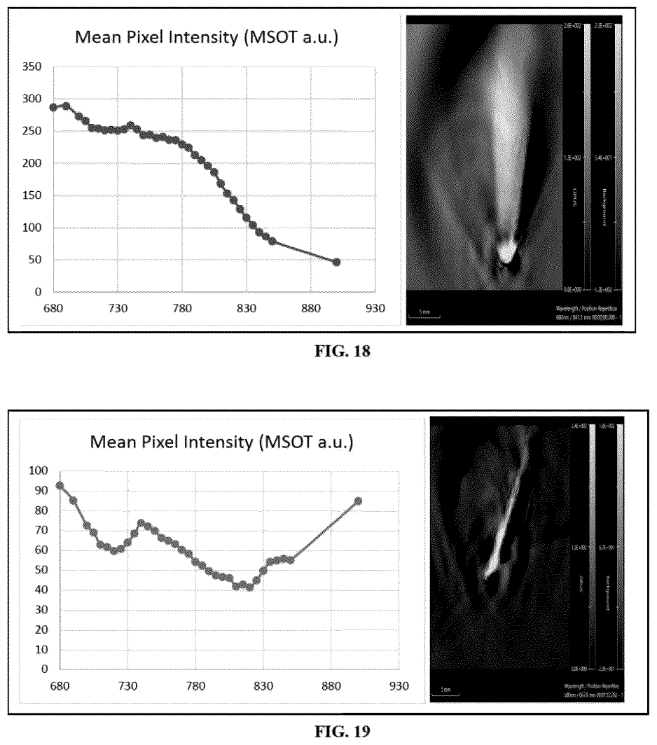

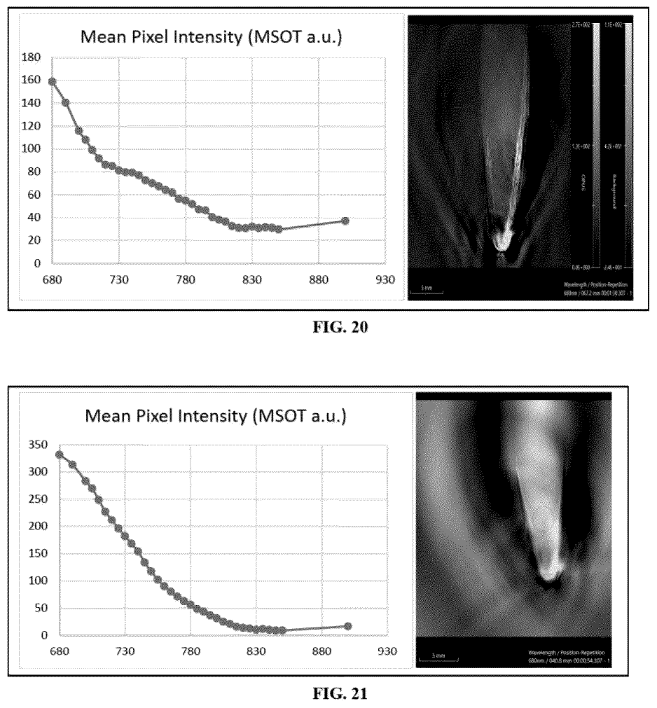

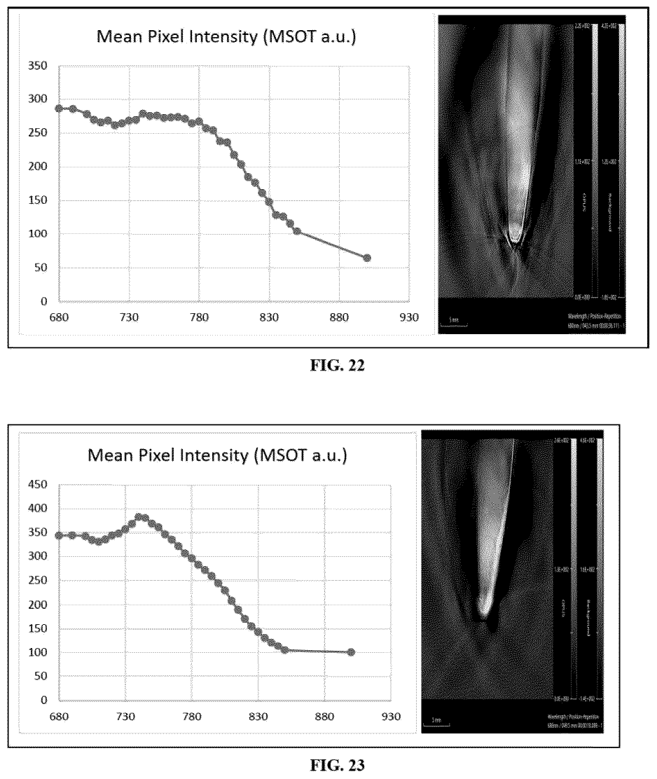

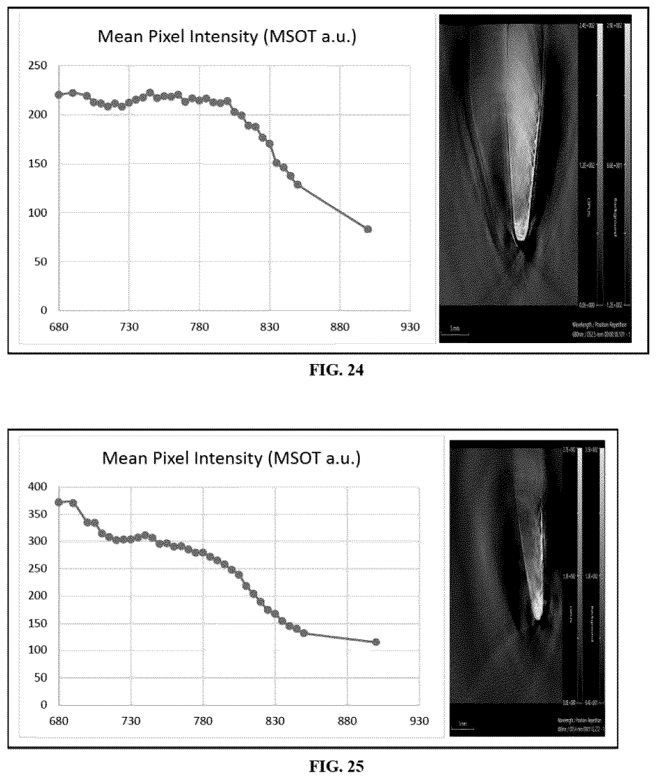

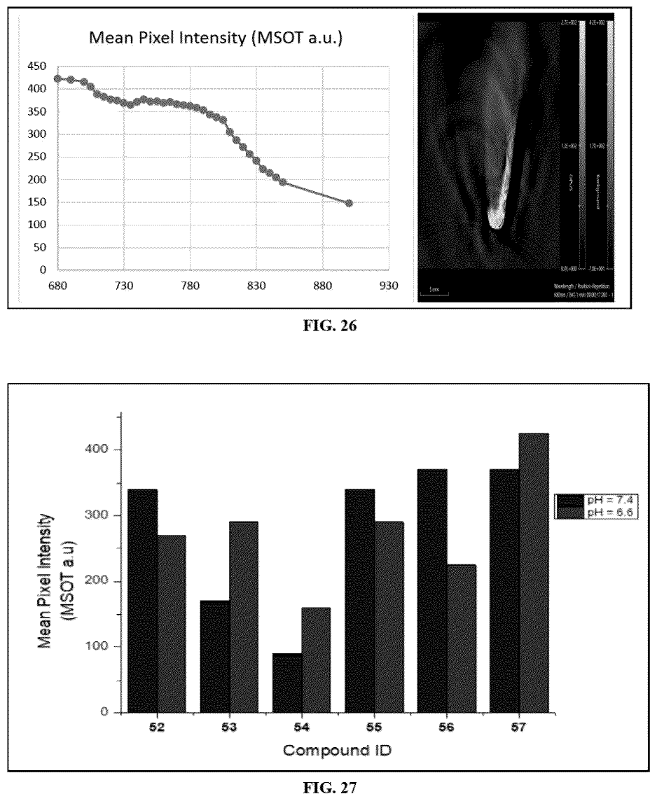

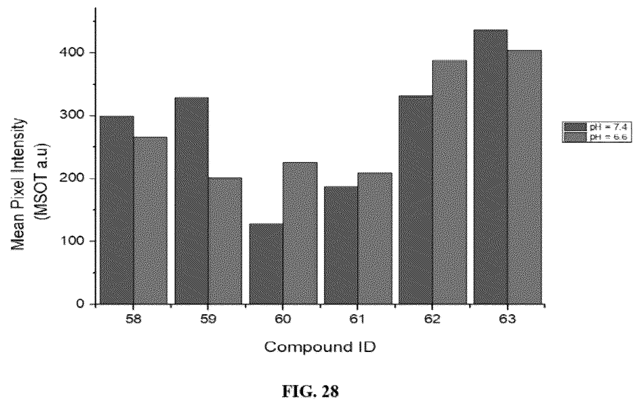

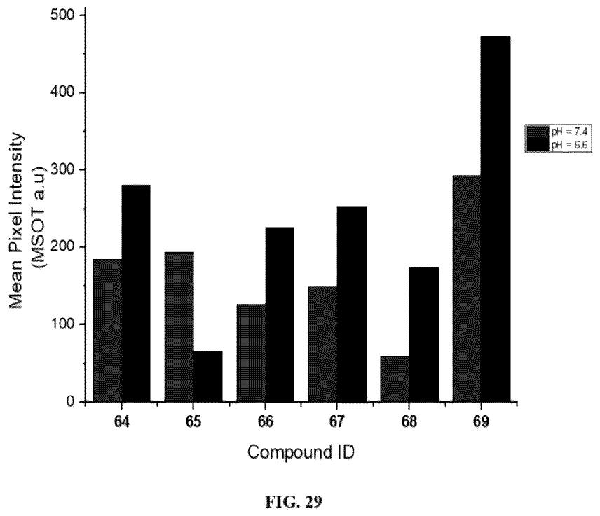

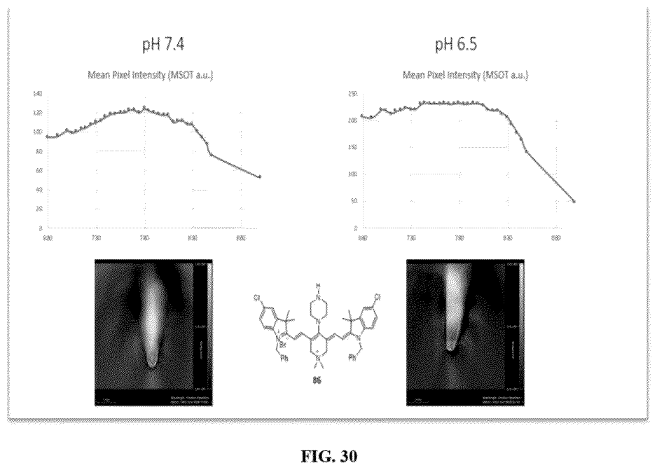

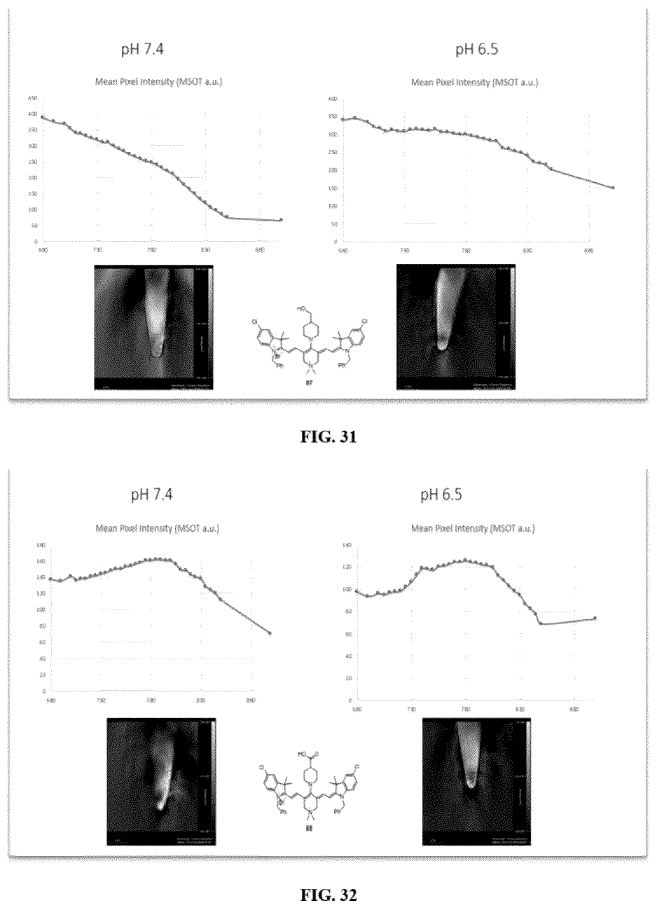

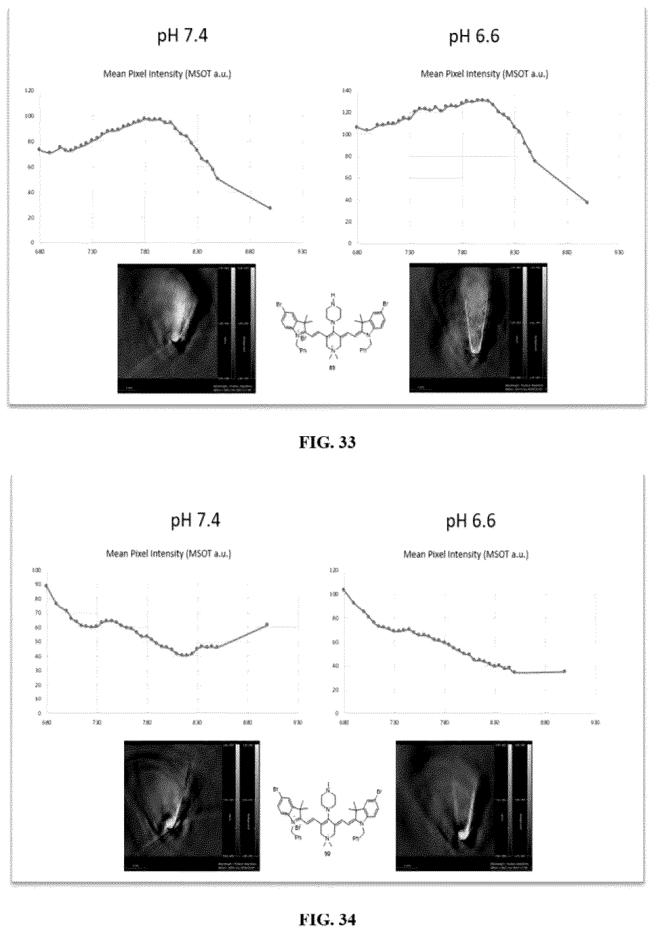

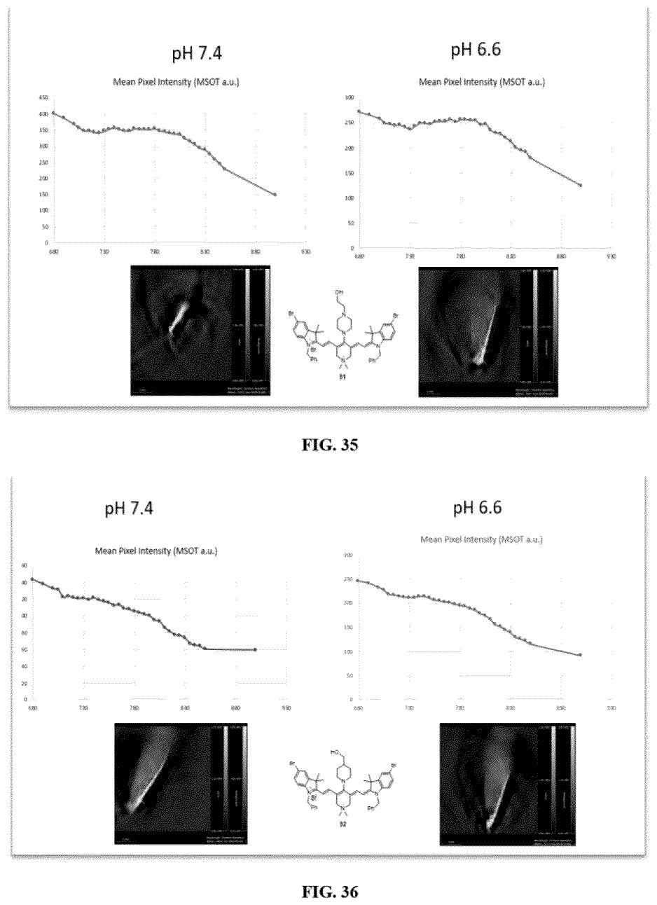

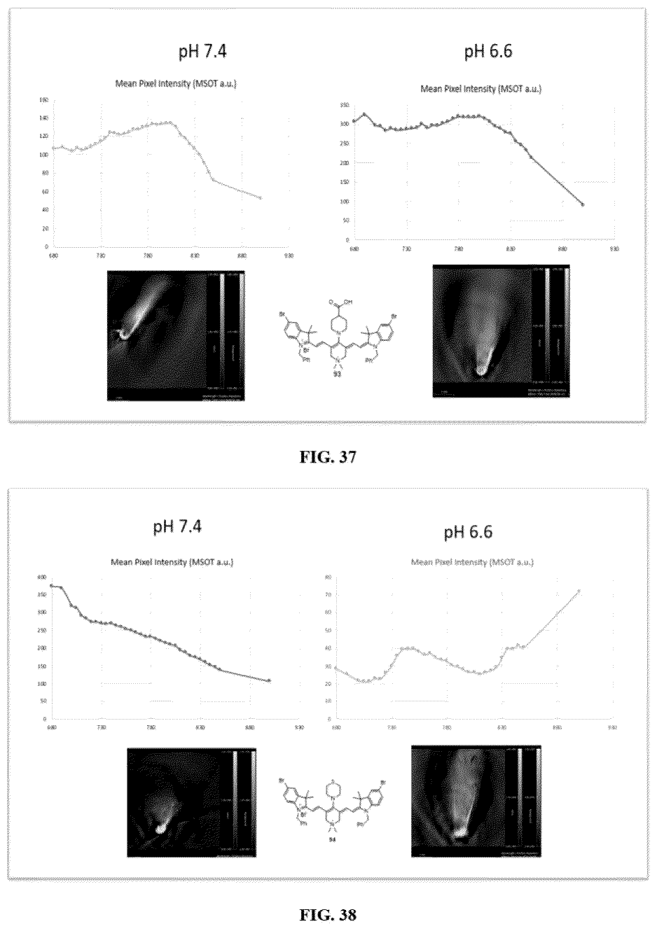

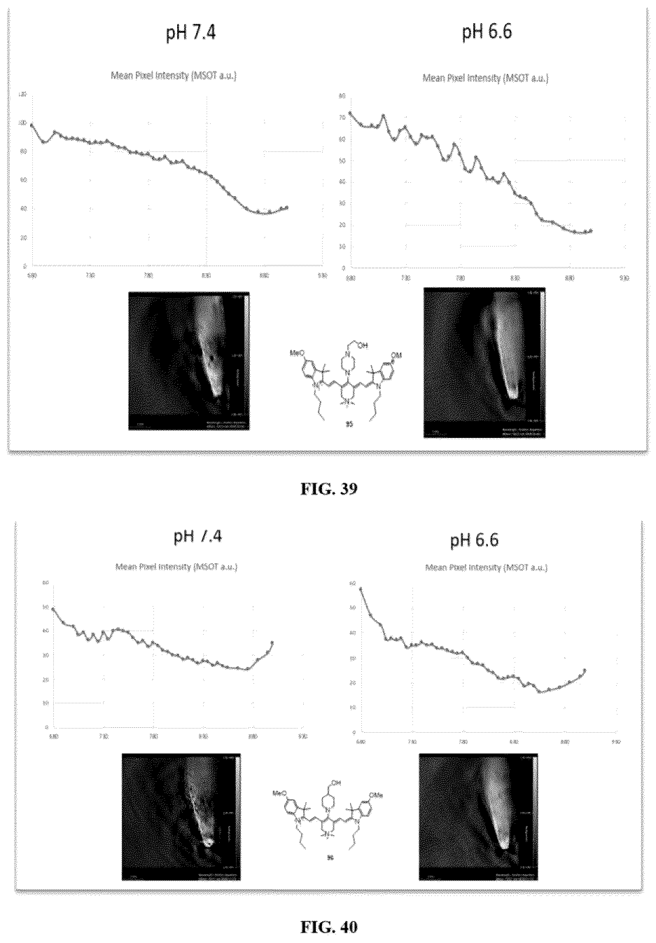

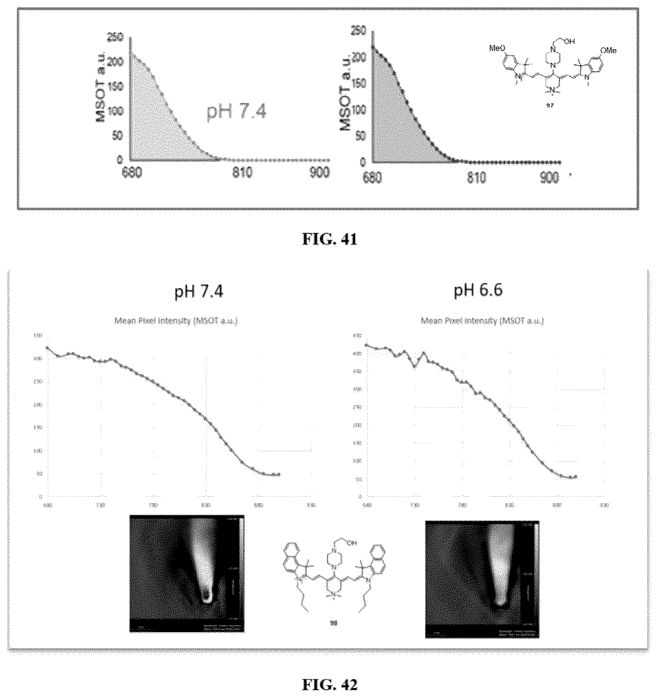

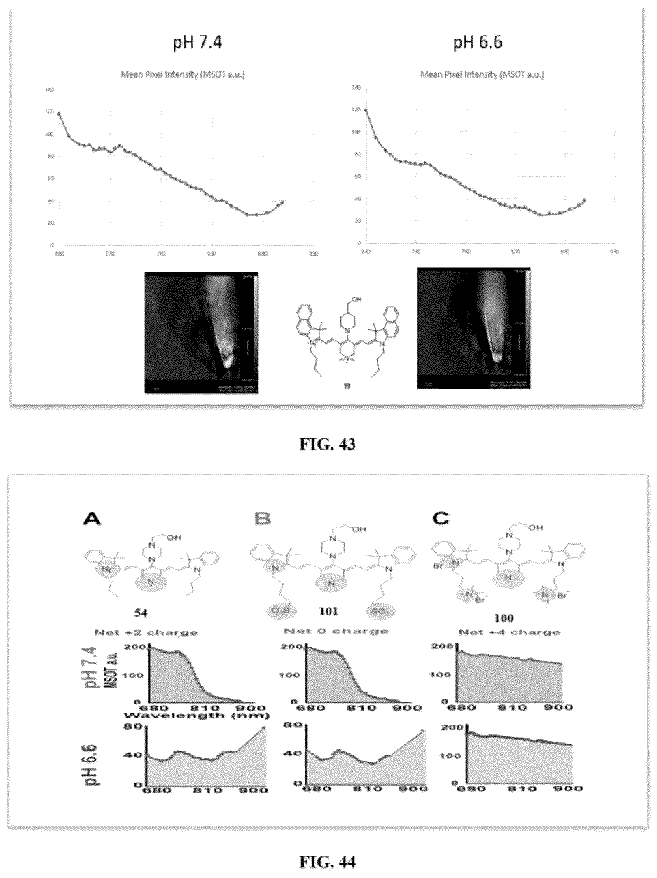

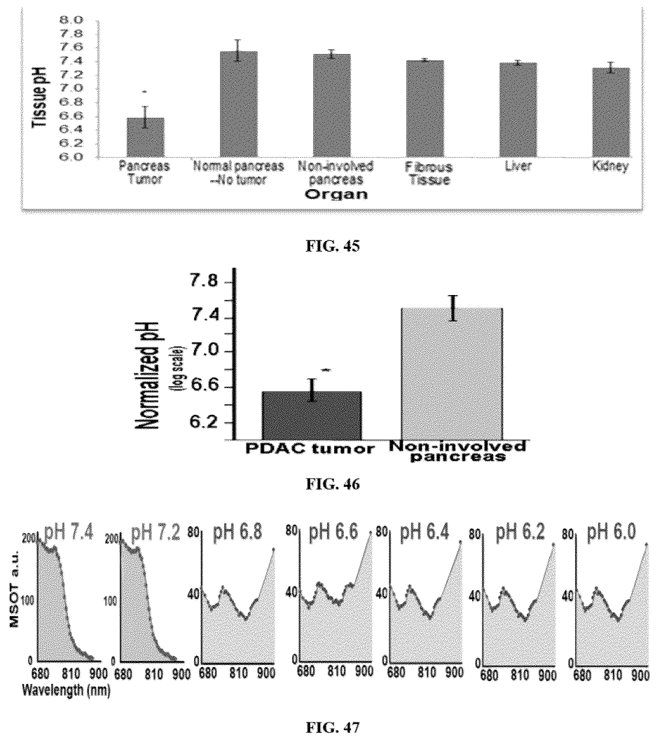

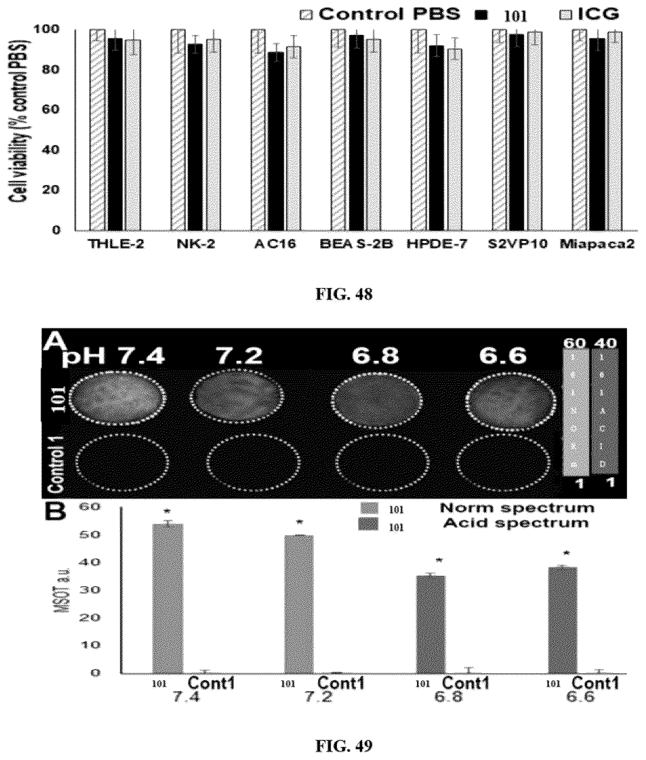

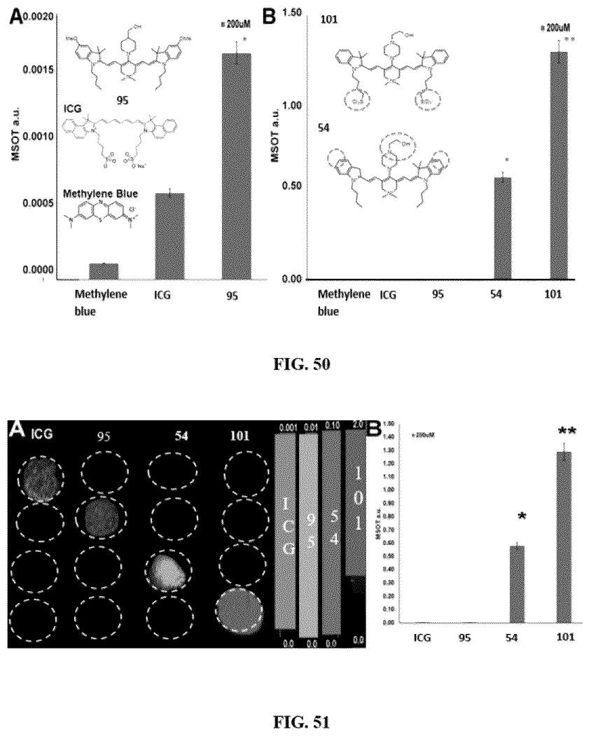

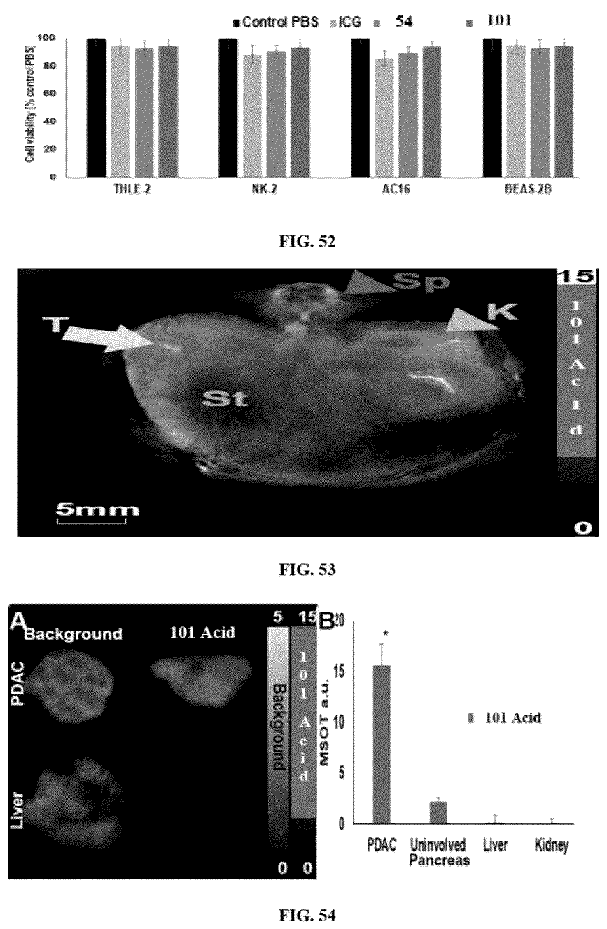

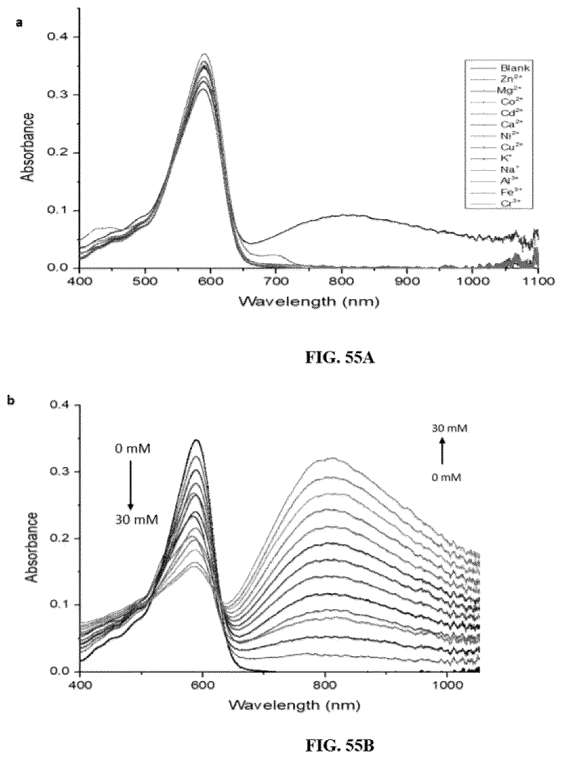

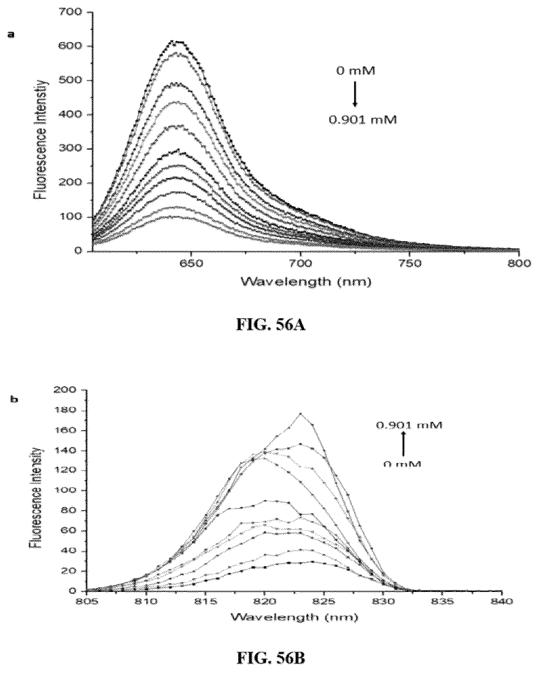

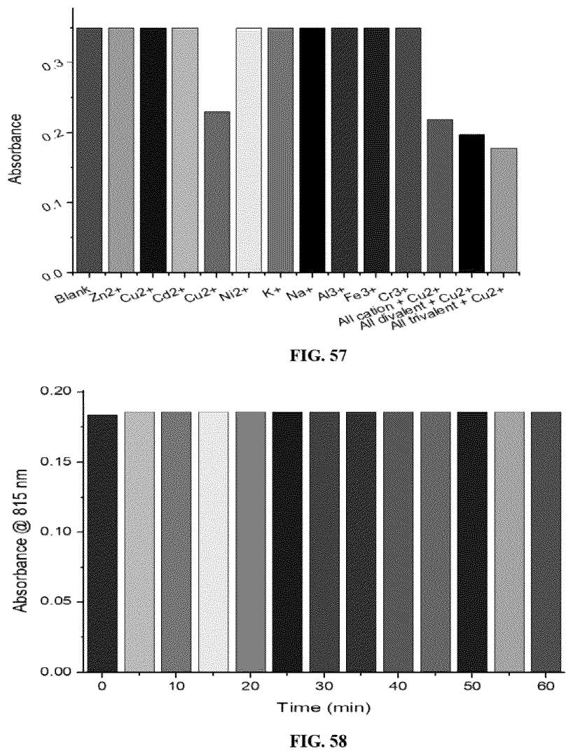

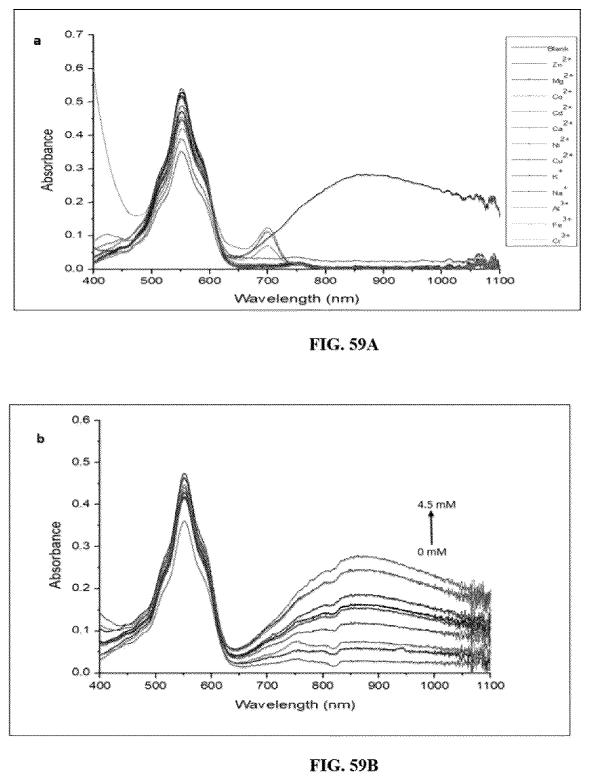

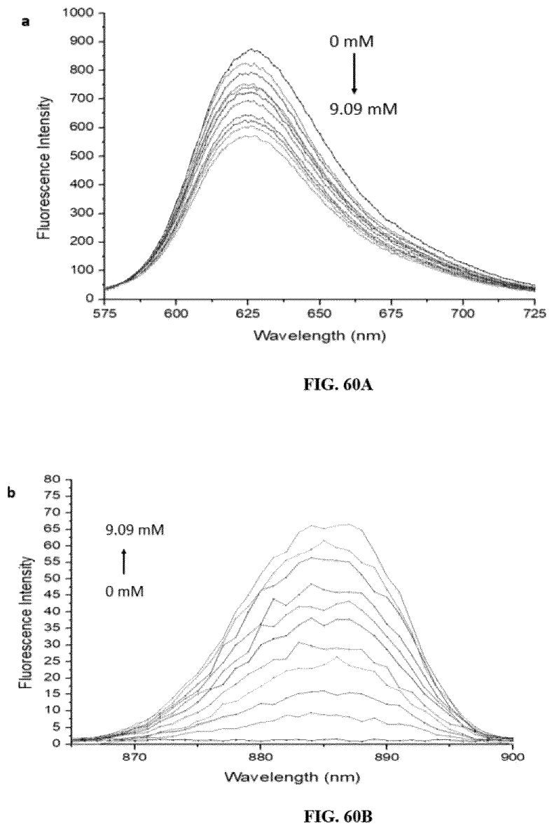

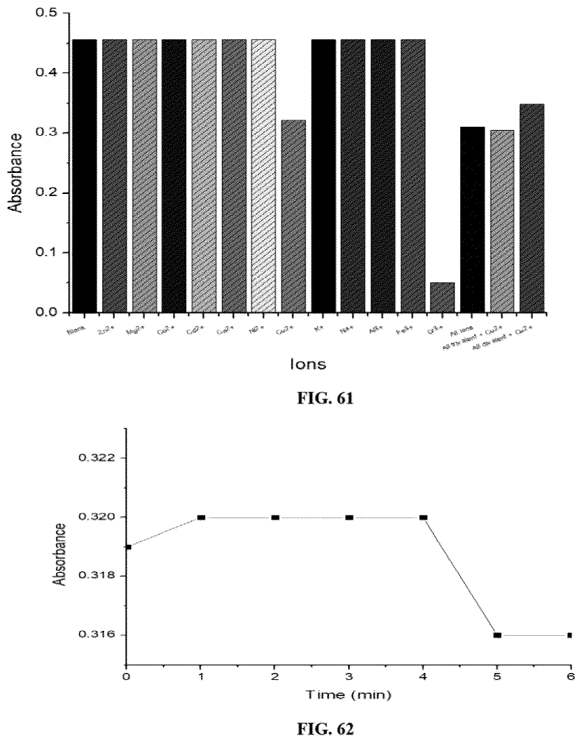

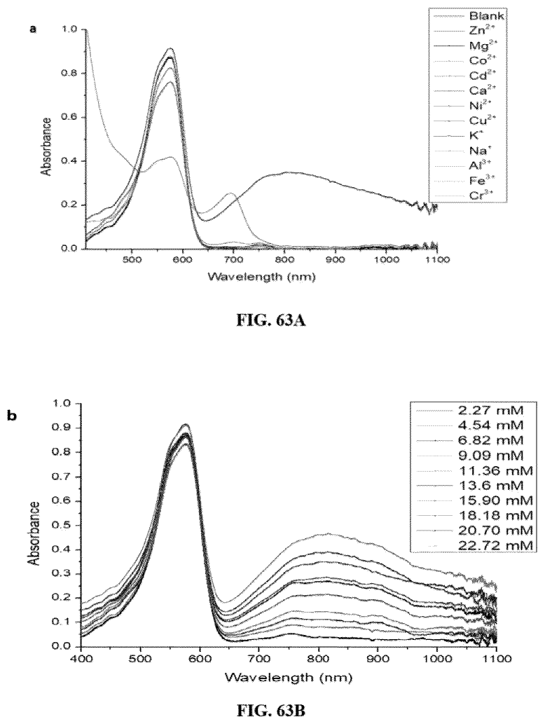

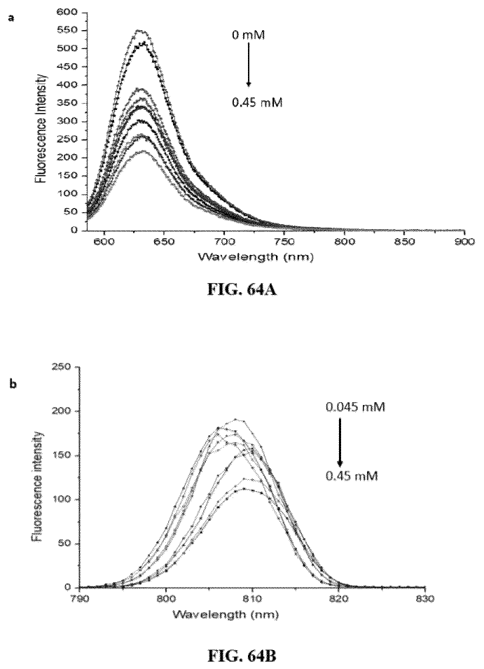

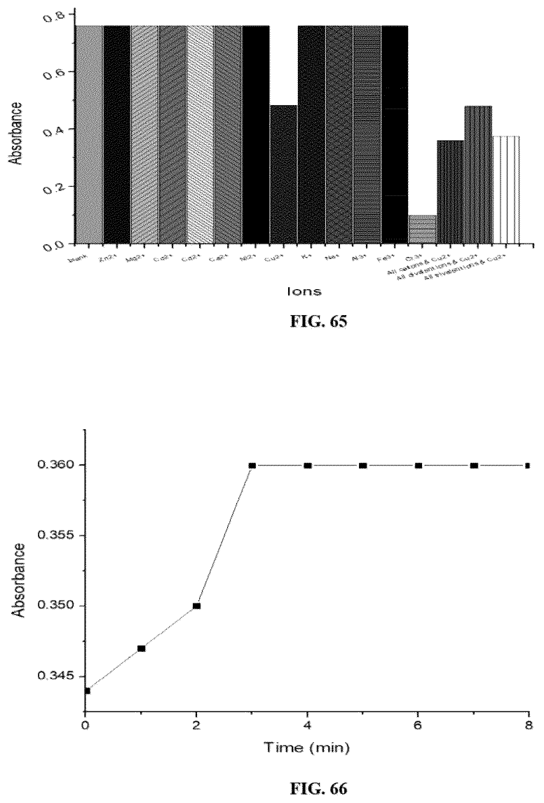

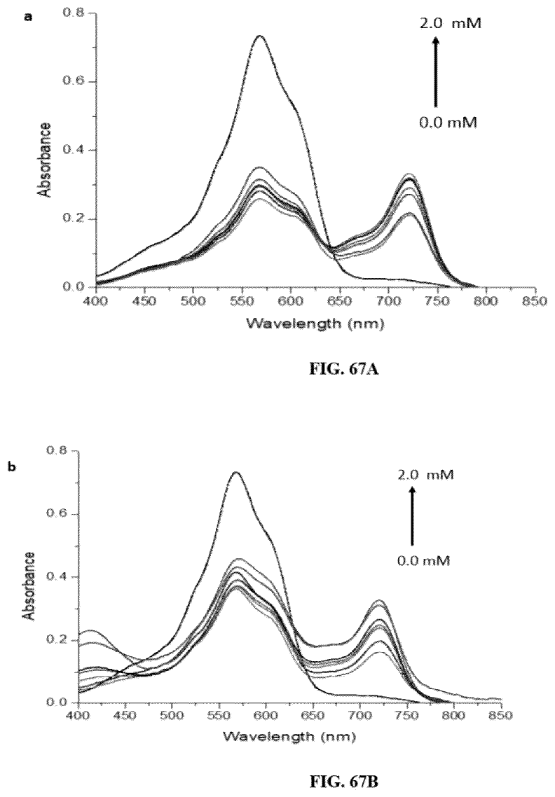

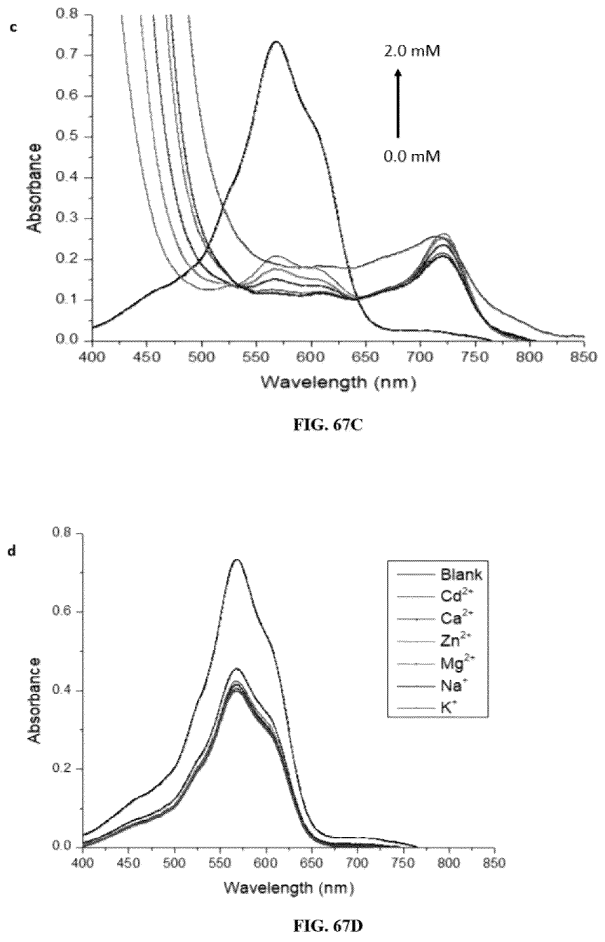

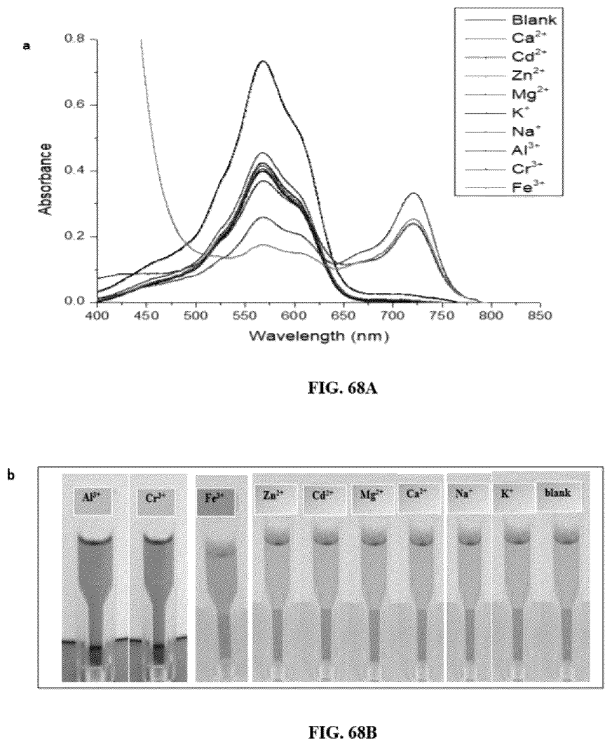

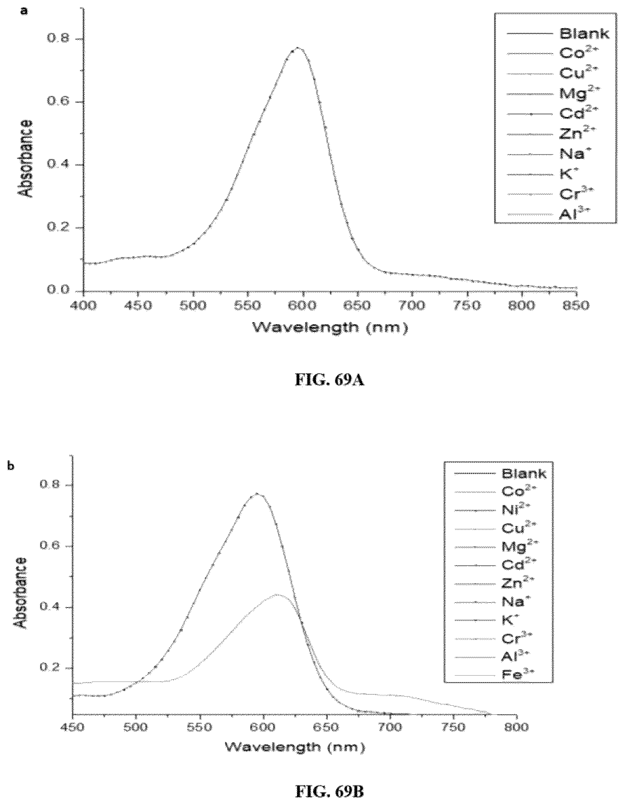

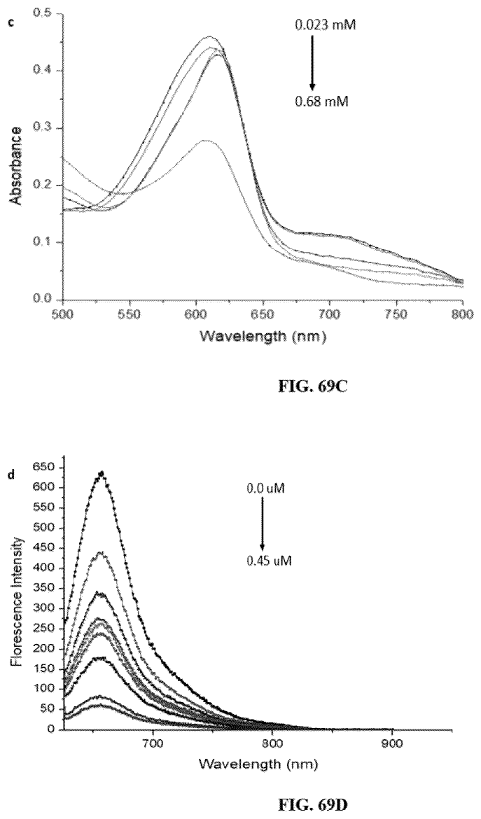

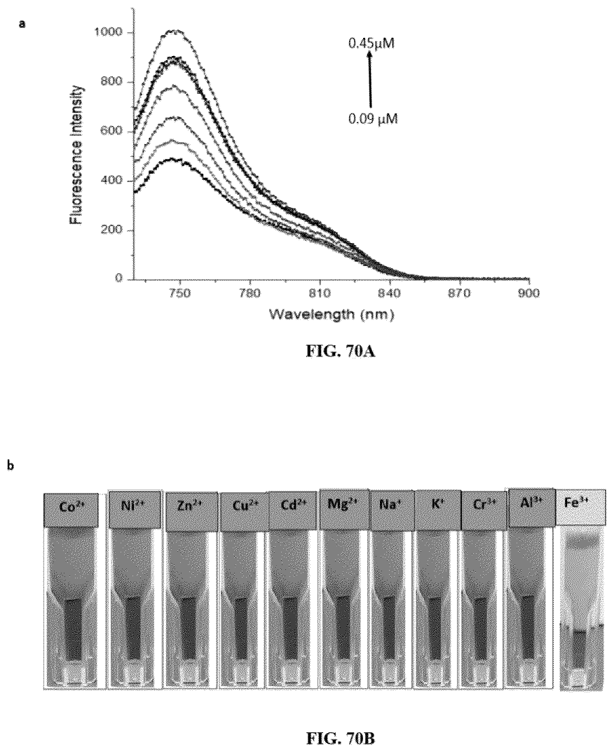

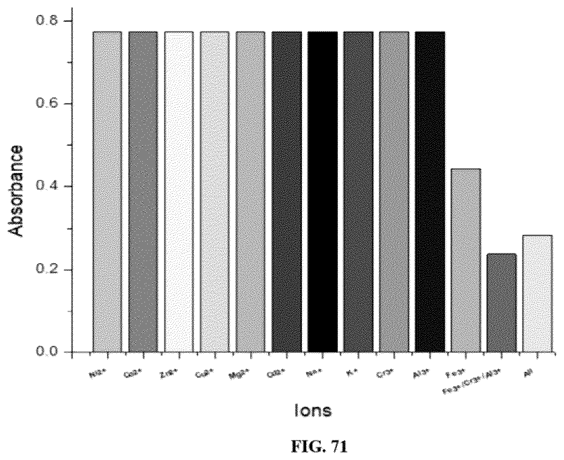

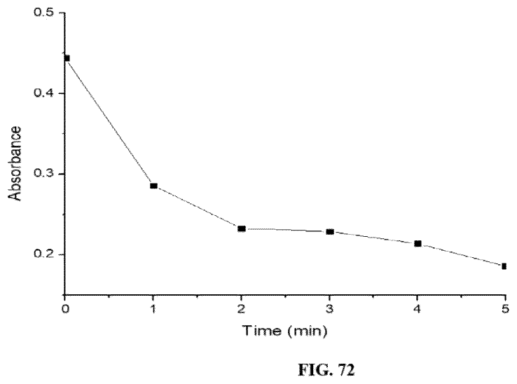

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprises” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋₈alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂—). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and lor more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and lor more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1carbon atom and 1heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified

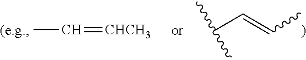

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and lor more heteroatoms within the parent chain (“heteroC₂₋₉ alkynyl”). In some embodiments, a heteroalkynyl group has 2 to 8 carbon atoms, at least one triple bond, and lor more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂-6 alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₄₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl.

Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R′, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{bb})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR^{bb} or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R′, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR)OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₄₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R′)₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted ammino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or dialkylamino, mono- or di-heteroalkylamino, mono- or di-arylamino, or mono- or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{c}c)OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, a chemical bond depicted: [symbol] represents either a single, double, or triple bond, valency permitting. By way of example,

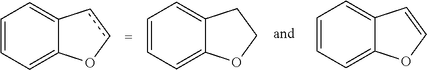

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}; C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Compounds disclosed herein may exist as one or more tautomers. Tautomers are interconvertible structural isomers that differ in the position of one or more protons or other labile atom. By way of example:

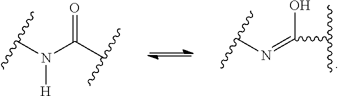

The prevalence of one tautomeric form over another will depend both on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

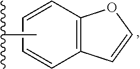

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

As used herein, “therapeutic” generally refers to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

The terms “treating” and “treatment” as used herein refer generally to obtaining a desired pharmacological and/or physiological effect. The effect may be prophylactic in terms of preventing or partially preventing a disease, symptom, or condition thereof.

As used interchangeably herein, “subject,” “individual,” or “patient,” refers to a vertebrate, preferably a mammal, more preferably a human. Mammals include, but are not limited to, murine, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “administration” refers to the injection of active agent on the subject.

Exemplary methods of administration include: intravenously (i.v.), intraperitoneally (i.p.), intratumorally (i.t.), or subcutaneously (s.c.) such as tissue ipsilateral (i.l.) to the tumor and tissue contralateral (c.l.) to the tumor.

Disclosed herein are cyanine compounds having the structure:

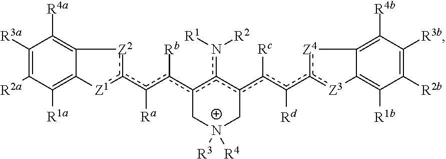

and pharmaceutically acceptable salts thereof, wherein
Z¹ is N—R^{na} and Z² is O, S, C(R^{z2})₂, NR^{na}, N═CR^{z2}, or C(R^{z2})═C(R^{z2}); or Z¹ is null and Z² is Z*—N(R^{na})—, wherein:
Z* is N═CR^{z2}, C(R^{z2})═C(R^{z2}); O, S, or C(R^{z2})₂;
R^{na} is in each case independently selected from C₁₋₈alkyl, optionally substituted one or more times by aryl, heteroaryl, COOH, OH, NH₂, NHCH₃, N(CH₃)₂, N(CH₃)₃, PO₃H₂, SO₃H,
R^{z2} is in case independently selected from F, Cl, Br, I, NO₂, CN, R^{z2*}, OR^{z2*}, N(R^{z2*})₂, SO₃R^{z2*}, SO₂R^{z2*}, SO₂N(R^{z2*})₂, C(O)R^{z2*}; C(O)OR^{z2*}, OC(O)R^{z2*}; C(O)N(R^{z2*})₂, N(R^{z2*})C(O)R^{z2*}, OC(O)N(R^{z2*})₂, N(R^{z2*})C(O)N(R^{z2*})₂, wherein R^{z2*} is in each case independently selected from hydrogen, C₁₋₈alkyl, aryl, C₁₋₈heteroaryl, C₃₋₈cycloalkyl, or C₁₋₈heterocyclyl;
Z³ is N—R^{nb} and Z⁴ is O, S, C(R^{z4})₂, NR^{nb}, N═CR^{z4}, or C(R^{z4})═C(R^{z4}); or Z³ is null and Z⁴ is Z** —N(R^{nb})—, wherein:
Z** is N═CR^{z4}, C(R^{z4})═C(R^{z4}), O, S, or C(R^{z4})₂;
R^{nb} is in case independently selected from C₁₋₈alkyl, optionally substituted one or more times by aryl, heteroaryl, COOH, OH, NH₂, NHCH₃, N(CH₃)₂, N(CH₃)₃, PO₃H₂, SO₃H;
R^{z4} is in case independently selected from F, Cl, Br, I, NO₂, CN, R^{z4*}, OR^{z4*}, N(R^{z4*})₂, SO₃R^{z4}* SO₂R^{z4*}, SO₂N(R^{z4*})₂, C(O)R^{z4*}; C(O)OR^{z4*}, OC(O)R^{z4*}; C(O)N(R^{z4*})₂, N(R^{z4*})C(O)R^{z4*}, OC(O)N(R^{z4*})₂, N(R^{z4*})C(O)N(R^{z4*})₂, wherein R^{z4*} is in each case independently selected from hydrogen, C₁₋₈alkyl, aryl, C₁₋₈heteroaryl, C₃₋₈cycloalkyl, or C₁₋₈heterocyclyl;
R^{1a} is F, Cl, Br, I, NO₂, CN, R^{1a*}, OR^{1a*}, SR^{1a*}, N(R^{1a*})₂, SO₃R^{1a*}, SO₂R^{1a*}, SO₂N(R^{1a*})₂, C(O)R^{1a*}; C(O)OR^{1a*}, OC(O)R^{1a*}; C(O)N(R^{1a*})₂, N(R^{1a*})C(O)R^{1a*}, OC(O)N(R^{1a*})₂, N(R^{1a*})C(O)N(R^{1a*})₂, wherein R^{1a*} is in each case independently selected from hydrogen, C₁₋₈alkyl, aryl, C₁₋₈heteroaryl, C₃₋₈cycloalkyl, or C₁₋₈heterocyclyl;
R^{2a} is F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, N(R^{2a*})C(O)N(R^{2a*})₂, wherein R^{2a}* is in each case independently selected from hydrogen, C₁₋₈alkyl, aryl, C₁₋₈heteroaryl, C₃₋₈cycloalkyl, or C₁₋₈heterocyclyl;
R^{3a} is F, Cl, Br, I, NO₂, CN, R^{3a*}, OR^{3a*}, SR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl;
R^{4a} is F, Cl, Br, I, NO₂, CN, R^{4a*}, OR^{4a*}, SR^{4a*}, N(R^{4a*})₂, SO₃R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a}* is in each case independently selected from hydrogen, C₁₋₈alkyl, aryl, C₁₋₈heteroaryl, C₃₋₈cycloalkyl, or C₁₋₈heterocyclyl;
R^{1b} is F, Cl, Br, I, NO₂, CN, R^{1b*}, OR^{1b*}, SR^{1b*}, N(R^{1b*})₂, SO₃R^{1b*}, SO₂R^{1b*}, SO₂N(R^{1b*})₂, C(O)R^{1b*}; C(O)OR^{1b*}, OC(O)R^{1b*}; C(O)N(R^{1b*})₂, N(R^{1b*})C(O)R^{1b*}, OC(O)N(R^{1b*})₂, N(R^{1b*})C(O)N(R^{1b*})₂, wherein R^{1b*} is in each case independently selected from hydrogen, C₁₋₈alkyl, aryl, C₁₋₈heteroaryl, C₃₋₈cycloalkyl, or C₁₋₈heterocyclyl;
R^{2b} is F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b}*; C(O)OR^{2b*}, OC(O)R^{2b}*; C(O)N(R^{2b*})₂, N(R^{2b*})C(O)R^{2b*}, OC(O)N(R^{2b*})₂, N(R^{2b*})C(O)N(R^{2b*})₂, wherein R^{2b*} is in each case independently selected from hydrogen, C₁₋₈alkyl, aryl, C₁₋₈heteroaryl, C₃₋₈cycloalkyl, or C₁₋₈heterocyclyl;
R^{3b} is F, Cl, Br, I, NO₂, CN, R^{3b*}, OR^{3b*}, SR^{3b*}, N(R^{3b*})₂, SO₃R^{3b*}, SO₂R^{3b*}, SO₂N(R^{3b*})₂, C(O)R^{3b}*; C(O)OR^{3b*}, OC(O)R^{3b}*; C(O)N(R^{3b*})₂, N(R^{3b*})C(O)R^{3b*}, OC(O)N(R^{3b*})₂, N(R^{3b*})C(O)N(R^{3b*})₂, wherein R^{3b*} is in each case independently selected from hydrogen, C₁₋₈alkyl, aryl, C₁₋₈heteroaryl, C₃₋₈cycloalkyl, or C₁₋₈heterocyclyl;
R^{4b} is F, Cl, Br, I, NO₂, CN, R^{4b*}, OR^{4b*}, SR^{4b*}, N(R^{4b*})₂, SO₃R^{4b*}, SO₂R^{4b*}, SO₂N(R^{4b*})₂, C(O)R^{4b}*; C(O)OR^{4b*}, OC(O)R^{4b}*; C(O)N(R^{4b*})₂, N(R^{4b*})C(O)R^{4b*}, OC(O)N(R^{4b*})₂, N(R^{4b*})C(O)N(R^{4b*})₂, wherein R^{4b*} is in each case independently selected from hydrogen, C₁₋₈alkyl, aryl, C₁₋₈heteroaryl, C₃₋₈cycloalkyl, or C₁₋₈heterocyclyl;
R^{a} is F, Cl, Br, I, NO₂, CN, R^{a*}, OR^{a*}, SR^{a*}, N(R^{a*})₂, SO₂R^{a*}, SO₂N(R^{a*})₂, C(O)R^{a*}; C(O)OR^{a*}, OC(O)R^{a*}; C(O)N(R^{a*})₂, N(R^{a*})C(O)R^{a*}, OC(O)N(R^{a*})₂, N(R^{a*})C(O)N(R^{a*})₂, wherein R^{a*} is in each case independently selected from hydrogen, C₁₋₈alkyl, aryl, C₁₋₈heteroaryl, C₃₋₈cycloalkyl, or C₁₋₈heterocyclyl;
R^{b} is F, Cl, Br, I, NO₂, CN, R^{b*}, OR^{b*}, SR^{b*}, N(R^{b*})₂, SO₂R^{b*}, SO₂N(R^{b*})₂, C(O)R^{b*}; C(O)OR*, OC(O)R^{b*}; C(O)N(R^{b*})₂, N(R^{b*})C(O)R^{b*}, OC(O)N(R^{b*})₂, N(R^{b*})C(O)N(R^{b*})₂, wherein R^{b*} is in each case independently selected from hydrogen, C₁₋₈alkyl, aryl, C₁₋₈heteroaryl, C₃₋₈cycloalkyl, or C₁₋₈heterocyclyl;
R^{c} is F, Cl, Br, I, NO₂, CN, R^{c*}, OR^{c*}, SR^{c*}, N(R^{c*})₂, SO₂R^{c*}, SO₂N(R^{c*})₂, C(O)R^{c*}; C(O)OR^{c*}, OC(O)R^{c*}; C(O)N(R^{c*})₂, N(R^{c*})C(O)R^{c*}, OC(O)N(R^{c*})₂, N(R^{c*})C(O)N(R^{c*})₂, wherein R^{c*} is in each case independently selected from hydrogen, C₁₋₈alkyl, aryl, C₁₋₈heteroaryl, C₃₋₈cycloalkyl, or C₁₋₈heterocyclyl;
R^{d} is F, Cl, Br, I, NO₂, CN, R^{d*}, OR^{d*}, SR^{d*}, N(R^{d*})₂, SO₂R^{d*}, SO₂N(R^{d*})₂, C(O)R^{d*}; C(O)OR^{d*}, OC(O)R^{d*}; C(O)N(R^{d*})₂, N(R^{d*})C(O)R^{d*}, OC(O)N(R^{d*})₂, N(R^{d*})C(O)N(R^{d*})₂, wherein R^{d*} is in each case independently selected from hydrogen, C₁₋₈alkyl, aryl, C₁₋₈heteroaryl, C₃₋₈cycloalkyl, or C₁₋₈heterocyclyl;
R¹ and R² are independently selected from H, C₁₋₆alkyl, C₂₋₆alkenyl, C₂₋₆alkynyl aryl, C₃₋₆cycloalkyl, C₁₋₆heterocyclyl, or C₁₋₆heteroaryl, wherein R^{t} and R² can together form a ring; wherein one or both of R¹ and R² are substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene;
R³ and R⁴ are independently selected from C₁₋₃alkyl, and may together form a ring; wherein each is optionally substituted one or more times by -Q¹-OR^{y}, -Q¹-N(R^{y})₂, -Q¹-N(R^{y′})₃, ═O, aryl, -Q¹-SO₃R^{y}, -Q¹-PO₃(R^{y})₂, -Q¹-C(O)R^{y}, -Q¹-C(O)OR^{y}, -Q¹-C(O)N(R^{y})₂, wherein R^{y} is in each case independently selected from H, C₁₋₆alkyl, R^{y′} is independently selected from C₁₋₃alkyl, wherein any two or more of R^{y} and R^{y′} can together form a ring; and Q¹ is null or C₁₋₄alkylene.

In some embodiments, the compounds are characterized by the proviso that when Rt is H, R² is not phenyl.

The skilled person understands that the compounds disclosed herein may be depicted in one of several resonance forms, e.g.:

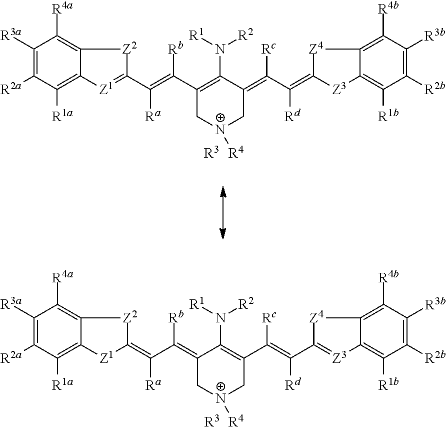

The depiction of one resonance form is intended to cover all possible other resonance forms as well.

The compounds disclosed herein may exist as a single geometric isomer, or may exist as a mixture of geometric isomers, e.g.:

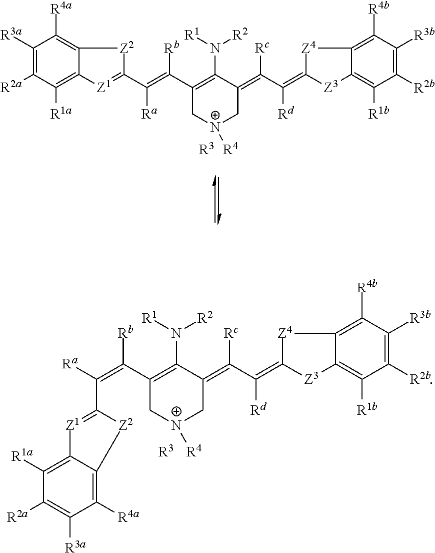

Unless specified explicitly to the contrary, the depiction of one geometric isomer is intended to cover any and all other geometric isomers, either as a single isomer or a mixture of isomers.

The compounds disclosed herein include quaternary nitrogen atoms, each having a formal charge of +1. Unless specified explicitly to the contrary, all compounds disclosed herein are electrically neutral, meaning that the sum total of cationic charges is equal to the sum total of anionic charges. In some instances, each of the corresponding anionic groups will be part of a functional group covalently bonded to the compound, e.g., R³=—CH₂CH₂SO₃H. In such instances, the compound may be designated as zwitterionically balanced. In other instances, at least some of the anionic groups will be supplied from exogenous anionic species, e.g., chloride anion, bromide anion, acetate anion, etc. In such cases, the compound may be designated as non-zwitterionically balanced.

In some embodiments, neither R¹ nor R² are an aryl ring, e.g., not a phenyl ring. In such cases R¹ and R² can be selected from H, C₁₋₆alkyl, C₂₋₆alkenyl, C₂₋₆alkynyl C₃₋₆cycloalkyl, or C₁₋₆heterocyclyl, wherein R¹ and R² can together form a ring; wherein one or both of R¹ and R² are substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene.

In yet further amendments, R¹ and R² are independently selected from C₁₋₆alkyl, C₂₋₆alkenyl, and C₂₋₆alkynyl and together form a ring substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene.

In certain instances, the cyanine compound is symmetrical, e.g., R^{1a}=R^{1b}, R^{2a}=R^{2b}, R^{3a}=R^{3b}, R^{4a}=R^{4b}, R^{a}=R^{d}, R^{b}=R^{c}, Z¹=Z³, and Z⁴=Z⁴. In some embodiments, the symmetrical cyanine is further characterized by R¹=R² (or R¹ and R² together form a symmetrical ring), or may be characterized by R³=R⁴ (or R³ and R⁴ together form a symmetrical ring).

In some embodiments, Z¹ is NR^{na}. In some instances when Z¹ is NR^{na}, Z² can be C(R²)₂, N═CR^{z2}, or C(R^{z2})═C(R^{z2}), e.g, a compound of formula:

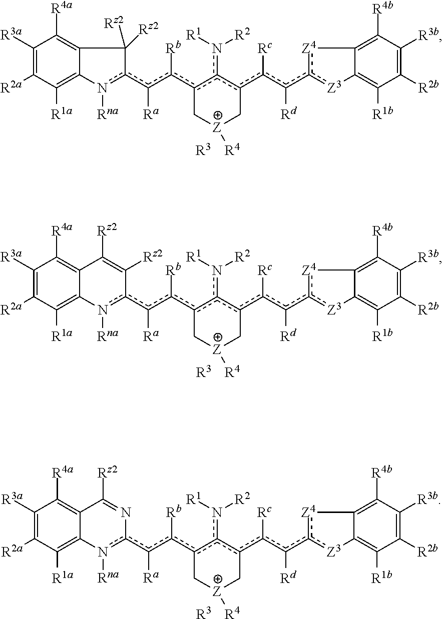

In other embodiments, Z¹ can be null. In some instances when Z¹ is null Z² is Z*—N(R^{na})—, wherein Z* is N═CR^{z2} or C(R^{z2})═C(R^{z2}), e.g., a compound of formula:

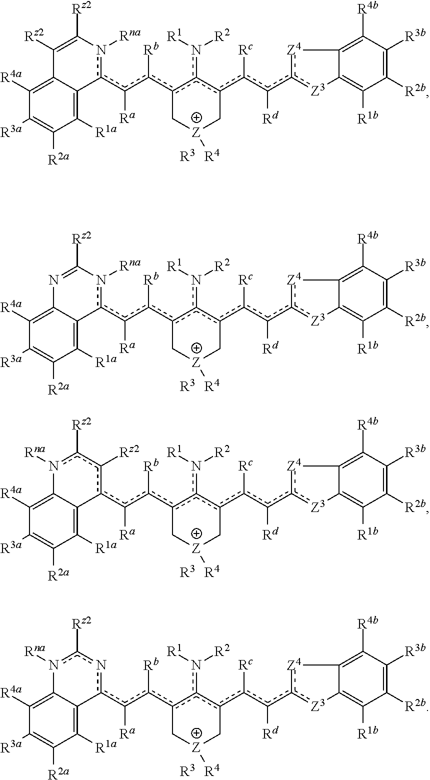

In certain embodiments Z³ is NR^{nb}. In some instance when Z³ is NR^{nb}, Z⁴ can be C(R^{z4})₂, N═CR^{z4} or C(R^{z4})═C(R^{z4}).

In other embodiments Z³ is null. In some instance when Z³ is null, Z⁴ is Z**—N(R^{nb})—, wherein Z** is N═CR^{z4} or C(R^{z4})═C(R^{z4}).

In certain embodiments, when Z¹ is NR^{na} Z² can be C(R^{z2})₂; preferred R^{z2} groups include methyl, ethyl, and when two R^{z2} groups together form a ring, e.g., a cyclopropyl ring.

In certain embodiments, when Z³ is NR^{nb} Z⁴ can be C(R^{z4})₂; preferred R^{z4} groups include methyl, ethyl, and when two R^{z2} groups together form a ring, e.g., a cyclopropyl ring.

In some embodiments, the heterocyclic systems at each end of the cyanine can be the same, for example Z¹ is NR^{na}, Z² is C(R^{z2})₂, Z³ is NR^{nb} and Z⁴ is a C(R^{z4})₂, i.e., a compound of formula:

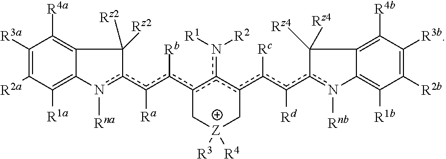

In other examples, Z³ can be NR^{nb} and Z⁴ is C(R^{z4})═C(R^{z4}). Similarly, Z¹ can be NR^{na} when Z² is C(R^{z2})═C(R^{z2}). In further embodiments, Z is NR^{na}, Z² is C(R^{z2})═C(R^{z2}), Z³ is NR^{nb} and Z⁴ C(R^{z4})═C(R^{z4}), i.e., a compound of formula:

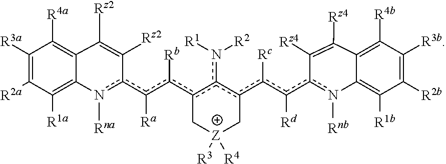

Other heterocyclic systems can also be used, for example benzoxazole, e.g., Z is NR^{na} and Z² is O or Z³ is NR^{nb} and Z⁴ is O; benzthiazole, e.g., Z is NR^{na} and Z² is S or Z³ is NR^{nb} and Z⁴ is S. Both heterocyclic systems may be benzoxazolyl, e.g., Z is NR^{na}, Z² is O, Z³ is NR^{nb}, and Z⁴ is O. Both heterocyclic systems may be benzthioazolyl, e.g., Z¹ is NR^{na}, Z² is S, Z³ is NR^{nb} and Z⁴ is S. Mixed benzoxazolyl/benzthiazolyl systems may also be used.

In preferred instances when Z¹ is NR^{na}, Z² will be C(CH₃)₂. Likewise, when Z³ is NR^{nb}, Z⁴ will be C(CH₃)₂.

Other heterocyclic systems include quinolinyl, e.g., Z³ is null and Z⁴ is Z**—N(R^{b})—, wherein Z** is C(R^{z4})═C(R^{z4}), or Z¹ is null and Z² is Z*—N(R^{na})—, wherein Z* is C(R^{z2})═C(R^{z2}).

Both heterocyclic systems may be quinolinyl, e.g., Z³ is null and Z⁴ is Z**—N(R^{nb})—, wherein Z** is C(R^{z4})═C(R^{z4}), Z¹ is null and Z² is Z*—N(R^{na})—, wherein Z* is C(R^{z2})═C(R^{z2}). In such systems, it is preferred that R^{z2} and R^{z4} are in each case H.

Other quinolinyl systems include those where Z³ is NR^{nb} and Z⁴ is C(R^{z4})═C(R^{z4}), or Z¹ is NR^{na} and Z² is C(R^{z2})═C(R^{z2}). In such systems, it is preferred that R^{z2} and R^{z4} are in each case H.

In preferred embodiments, the cyanine polyene portion of the compound is unsubstituted, e.g., R^{aa}, R^{b}, R^{c}, and R^{d} are each hydrogen.

In some instances, R^{1a}, R^{2a}, R^{3a}, and R^{4a} are each hydrogen or R^{1b}, R^{2b}, R^{3b}, and R^{4b} are each hydrogen. In some embodiments, the cyanine compound is unsubstituted, e.g., R^{a}, R^{b}, R^{c}, R^{d}, R^{1a}, R^{2a}, R^{3a}, R^{4a}, R^{1b}, R^{2b}, R^{3b}, and R^{4b} are each hydrogen.

In other embodiments, at least one of R^{1a}, R^{2a}, R^{3a}, and R^{4a} is not hydrogen. In the case of symmetrical cyanine compounds, the corresponding of R^{1b}, R^{2b}, R^{3b}, and R^{4b} will also not be hydrogen. Suitable substituents include electron withdrawing groups and electron donating groups. For example, at least one of R^{1a}, R^{2a}, R^{3a}, or R^{4a} are F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene.

In exemplary embodiments, R^{1a} is F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{2a}, R^{3a}, and R^{4a} are hydrogen.

In other embodiments, R^{2a} is F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{1a}, R^{3a}, and R^{4a} are hydrogen.

In other embodiments, R^{3a} is F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{1a}, R^{2a}, and R^{4a} are hydrogen.

In other embodiments, R^{4a} is F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{1a}, R^{2a}, and R^{3a} are hydrogen.

In other embodiments, R^{1a} and R^{2a} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{3a} and R^{4a} are hydrogen.

In other embodiments, R^{2a} and R^{3a} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{1a} and R^{4a} are hydrogen.

In other embodiments, R^{3a} and R^{4a} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene, and R^{1a} and R^{2a} are hydrogen.

In other embodiments, wherein R^{1a} and R^{3a} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q-PO₃H₂, and Q¹-CO₂H, wherein Q¹ is C₁₋₆alkylene, and R^{2a} and R^{4a} are hydrogen.

In other embodiments, R^{2a} and R^{4a} are independently selected from F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N^{m}(C₁₋₃alkyl)₃, Q¹-SO₃H, Q¹-PO₃H₂, and Q¹-CO₂H, wherein Q¹ is C₁₋₆alkylene, and R^{1a} and R^{3a} are hydrogen.

In the case of symmetrical cyanine compounds, any definitions applied to one of R^{1a}, R^{2a}, R^{3a}, and R^{4a} would be applied to the corresponding of R^{1b}, R^{2b}, R^{3b}, and R^{4b}. In the case of non-symmetrical cyanines, at least one of R^{1a}, R^{2a}, R^{3a}, and R^{4a} will be different than the corresponding of R^{1b}, R^{2b}, R^{3b}, and R^{4b}. For example, at least one of R^{1b}, R^{2b}, R^{3b}, or R^{4b} are F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene.

In exemplary embodiments, R^{1b} is F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q-SO₃H, QL-PO₃H₂, and Q¹-CO₂H, wherein Q¹ is C₁₋₆alkylene, and R^{2b}, R^{3b}, and R^{4b} are hydrogen.

In other embodiments, R^{2b} is F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b}, R^{3b}, and R^{4b} are hydrogen.

In other embodiments, R^{3b} is F, Cl, Br, I, CN, C_{1-3b}lkyl, C₁₋₃haloalkyl, QL-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b}, R^{2b}, and R^{4b} are hydrogen.

In other embodiments, R^{4b} is F, Cl, Br, I, CN, C_{1-3b}lkyl, C₁₋₃haloalkyl, QL-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b}, R^{2b}, and R^{3b} are hydrogen.

In other embodiments, R^{1b} and R^{2b} are independently selected from F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N^{m}(C_{1-3b}lkyl)₃, QL-SO₃H, Q¹-PO₃H₂, and QL-CO₂H, wherein Q¹ is C₁₋₆alkylene, and R^{3b} and R^{4b}are hydrogen.

In other embodiments, R^{2b} and R^{3b} are independently selected from F, Cl, Br, I, CN, C_{1-3b}lkyl, C₁₋₃haloalkyl, Q¹-OH, QL-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b} and R^{4b} are hydrogen.

In other embodiments, R^{3b} and R^{4b} are independently selected from F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b} and R^{2b}are hydrogen.

In other embodiments, wherein R^{1b} and R^{3b} are independently selected from F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{2b} and R^{4b} are hydrogen.

In other embodiments, R^{2b} and R^{4b} are independently selected from F, Cl, Br, I, CN, C_{1-3b}lkyl, C₁₋₃haloalkyl, Q¹-OH, Q¹-OC_{1-3b}lkyl, Q¹-NH₂, Q¹-NHC_{1-3b}lkyl, Q¹-N(C_{1-3b}lkyl)₂, Q¹-N⁺(C_{1-3b}lkyl)₃, Q¹-SO₃H, Q¹-PO₃H₂, and Q¹-CO₂H, wherein Q¹ is C₁₋₆alkylene, and R^{1b} and R^{3b} are hydrogen.

Exemplary substituents include F, Cl, OH, OCH₃, COOH, SO₃H, NH₂, N(CH₃)₂, NHCH₃, N⁺(CH₃)₃, CH₂N⁺(CH₃)₃, CH₂CH₂N⁺(CH₃)₃, CH₂CH₂CH₂N⁺(CH₃)₃, CH₂CH₂CH₂CH₂N⁺(CH₃)₃, CH₂COOH, CH₂CH₂COOH, CH₂CH₂CH₂COOH, or CH₂CH₂CH₂CH₂COOH.

The following sub-embodiments also form part of the disclosure:
R^{2a} is Q¹-OC₁₋₃alkyl and each of R^{1a}, R^{3a}, and R^{4a} are hydrogen.
R^{4a} is Q¹-OC₁₋₈alkyl and each of R^{1a}, R^{2a}, and R^{3a} are hydrogen.
R^{1a} Q¹-OC₁₋₃alkyl and each of R^{2a}, R^{3a}, and R^{4a} are hydrogen.
R^{4a} is Q¹-OC₁₋₃alkyl and each of R^{1a}, R^{2a}, and R^{3a} are hydrogen.
R^{1a} and R^{3a} are independently selected from Q¹-OC₁₋₃alkyl, and each of R^{2a} and R^{4a} are hydrogen.
R^{2a} and R^{2d} are independently selected from Q¹-OC₁₋₃alkyl and each of R^{1a} and R^{3a} are hydrogen.
R^{1a} and R^{2a} are independently selected from Q¹-OC₁₋₃alkyl and each of R^{3a} and R^{4a} are hydrogen.
R^{2a} and R^{3a} are independently selected from Q¹-OC₁₋₃alkyl and each of R^{1a} and R^{4a} are hydrogen.
R^{3a} and R^{4a} are independently selected from Q¹-OC₁₋₃alkyl and each of R^{1a} and R^{2a} are hydrogen.
R^{2a} is F, Cl, or Br and each of R^{1a}, R^{3a}, and R^{4a} are hydrogen.
R^{4a} is F, Cl, or Br and each of R^{1a}, R^{2a}, and R^{3a} are hydrogen.
R^{1a} is F, Cl, or Br and each of R^{2a}, R^{3a}, and R^{4a} are hydrogen.
R^{4a} is F, Cl, or Br and each of R^{1a}, R^{2a}, and R^{3a} are hydrogen.
R^{1a} and R^{3a} are independently selected from F, Cl, or Br, and each of R^{2a} and R^{4a} are hydrogen.
R^{2a} and R^{2d} are independently selected from F, Cl, or Br and each of R^{1a} and R^{3a} are hydrogen.
R^{1a} and R^{2a} are independently selected from F, Cl, or Br and each of R^{3a} and R^{4a} are hydrogen.
R^{2a} and R^{3a} are independently selected from F, Cl, or Br and each of R^{1a} and R^{4a} are hydrogen.
R^{3a} and R^{4a} are independently selected from F, Cl, or Br and each of R^{1a} and R^{2a} are hydrogen.
R^{2b} is Q¹-OC₁₋₃alkyl and each of R^{1b}, R^{3b}, and R^{4b} are hydrogen.
R^{4b} is Q¹-OC₁₋₃alkyl and each of R^{1b}, R^{2b}, and R^{3b} are hydrogen.
R^{1b} Q¹-OC₁₋₃alkyl and each of R^{2b}, R^{3b}, and R^{4b} are hydrogen.
R^{4b} is Q¹-OC₁₋₃alkyl and each of R^{1b}, R^{2b}, and R^{3b} are hydrogen.
R^{1b} and R^{3b} are independently selected from Q¹-OC₁₋₃alkyl, and each of R^{2b} and R^{4b} are hydrogen.
R^{2b} and R^{2d} are independently selected from Q¹-OC₁₋₃alkyl and each of R^{1b} and R^{3b} are hydrogen.
R^{1b} and R^{2b} are independently selected from Q¹-OC₁₋₃alkyl and each of R^{3b} and R^{4b} are hydrogen.
R^{2b} and R^{3b} are independently selected from Q¹-OC₁₋₃alkyl and each of R^{1b} and R^{4b} are hydrogen.
R^{3b} and R^{4b} are independently selected from Q¹-OC₁₋₃alkyl and each of R^{1b} and R^{2b} are hydrogen.
R^{2b} is F, Cl, or Br and each of R^{1a}, R^{3b}, and R^{4b} are hydrogen.
R^{4b} is F, Cl, or Br and each of R^{1b}, R^{2b}, and R^{3b} are hydrogen.
R^{1b} is F, Cl, or Br and each of R^{2b}, R^{3b}, and R^{4b} are hydrogen.
R^{4b} is F, Cl, or Br and each of R^{1a}, R^{2b}, and R^{3b} are hydrogen.
R^{1b} and R^{3b} are independently selected from F, Cl, or Br, and each of R^{2b} and R^{4b} are hydrogen.
R^{2b} and R^{2d} are independently selected from F, Cl, or Br and each of R^{1b} and R^{3b} are hydrogen.
R^{1b} and R^{2b} are independently selected from F, Cl, or Br and each of R^{3b} and R^{4b} are hydrogen.
R^{2b} and R^{3b} are independently selected from F, Cl, or Br and each of R^{1b} and R^{4b} are hydrogen.
R^{3b} and R^{4b} are independently selected from F, Cl, or Br and each of R^{1b} and R^{2b} are hydrogen.

In some embodiments, R^{na} is an optionally substituted alkylene group, for example R^{na} can be —(CH₂)ₙₐ—X^{a}, wherein na is 1, 2, 3, 4, 5, 6, 7, or 8, and X^{a} is aryl, heteroaryl, H, COOH, PO₃H₂, SO₃H, OH, NH₂, NHCH₃, N(CH₃)₂, N(CH₃)₃. For example, R^{na} can be —(CH₂)₂—X^{a}, —(CH₂)₃—X^{a}, —(CH₂)₄—X^{a}, —(CH₂)₅—X^{a}, wherein X^{a} is phenyl, H, COOH, PO₃H₂, SO₃H, or N(CH₃)₃.

In some embodiments, R^{nb} is —(CH₂)_{nb}—X^{b}, wherein nb is 1, 2, 3, 4, 5, 6, 7, or 8, and X^{b} is aryl, heteroaryl, H, COOH, PO₃H₂, SO₃H, OH, NH₂, NHCH₃, N(CH₃)₂, N(CH₃)₃. For example, R^{nb} is —(CH₂)₂—X^{b}, —(CH₂)₃—X^{b}, —(CH₂)₄—X^{b}, —(CH₂)₅—X^{b}, wherein X^{b} is phenyl, H, COOH, PO₃H₂, SO₃H, or N(CH₃)₃.

In examples of symmetrical cyanine compounds, R^{na} and R^{nb} are the same, and can be selected from methyl, ethyl, propyl, butyl, benzyl, —(CH₂)₃—COOH, —(CH₂)₃—PO₃H₂, —(CH₂)₃—N(CH₃)₃, —(CH₂)₃-Ph, and —(CH₂)₃—SO₃H.

The nitrogen atom bearing the R¹ and R² substituents may be designated the “meso nitrogen.” In some embodiments, the meso nitrogen can be a secondary nitrogen, e.g., R¹ is H and R² is C₁₋₈alkyl, C₃₋₈cycloalkyl, or C₁₋₈heterocyclyl, substituted with one or more groups selected from -Q-OR^{z}, -Q-SR^{z}, -Q-N(R^{z})₂, -Q-N(R^{z′})₃, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene. Exemplary R² groups include (CH₂)_{nc}X^{c} wherein nc is 1, 2, 3, 4, 5, 6, 7, or 8, and X is H, CONH₂, COOH, PO₃H₂, SO₃H, SH, SCH₃, OH, OCH₃, NH₂, NHCH₃, N(CH₃)₂, or N(CH₃)₃. In other examples, R² is (CH₂)_{nc}X^{c} wherein nc is 1, 2, 3, 4, 5, 6, 7, or 8, and X is N(R^{2a})(R^{2b}), wherein R^{2a} is H or C₁₋₈alkyl, R^{2b} is H or C₁₋₈alkyl, wherein R^{2a} and R^{2b} may together form a ring.

In other embodiments, the meso nitrogen may be part of a cyclic system. For example, R¹ and R², together with the meso nitrogen can form a ring having the formula:

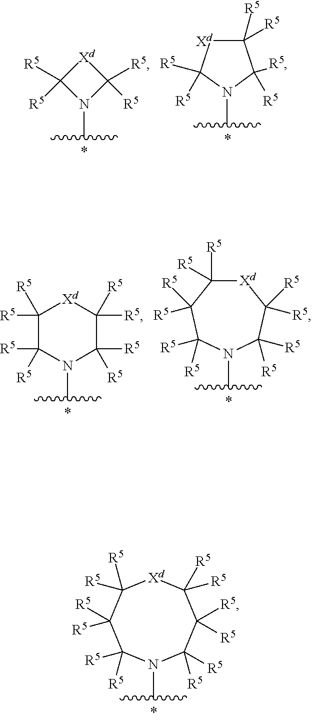

wherein the * represents the bond to the cyanine system,
R⁵ is in each case independently selected from F, Cl, Br, I, NO₂, CN, R^{5a*}, OR^{5a*}, N(R^{5a*})₂, SO₃R^{5a*}, SO₂R^{5a*}, SO₂N(R^{5a*})₂, C(O)R^{5a*}; C(O)OR^{5a*}, OC(O)R^{5a*}; C(O)N(R^{5a*})₂, N(R^{5a*})C(O)R^{5a*}, OC(O)N(R^{5a*})₂, N(R^{5a*})C(O)N(R^{5a*})₂, wherein R^{5a*} is in each case independently selected from hydrogen, C₁₋₈alkyl, aryl, C₁₋₈heteroaryl, C₃₋₈cycloalkyl, or C₁₋₈heterocyclyl;
X^{d} is null, O, S, C(R⁵)₂, or NR⁵,
wherein two geminal R⁵ groups may together form an oxo,
two or more R⁵ groups may together form a ring, and
wherein two adjacent R⁵ groups may together form a double bond.

In some embodiments when two or more R⁵ groups form a ring, the ring may have the structure:

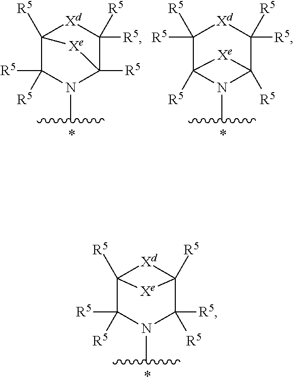

wherein X^{e} is null, O, S, NR⁵, or a C₁₋₄alkylene group having the formula: —(C(R⁵)₂)ₙₑ, wherein ne is 1, 2, 3, or 4. Preferred ring systems include:

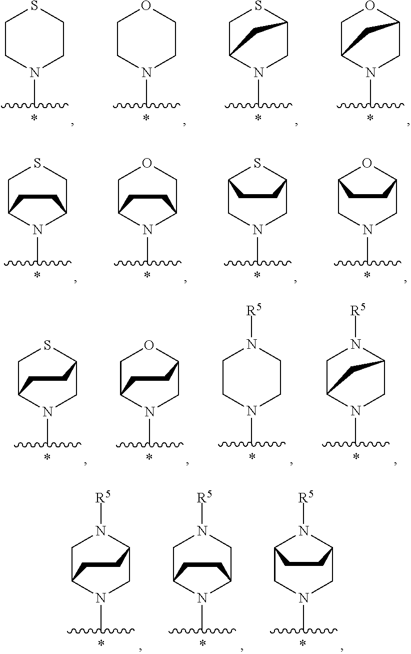

wherein R⁵ is H or —(CH₂)_{nf}, X^{c}, wherein nf is 1, 2, 3, 4, 5, or 5, and X is H, CONH₂, COOH, PO₃H₂, SO₃H, SH, SCH₃, OH, OCH₃, NH₂, NHCH₃, N(CH₃)₂, or N(CH₃)₃.

In some embodiments, the ring system can be:

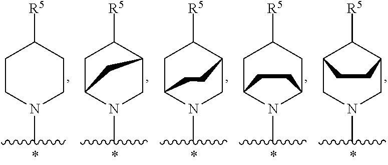

wherein R⁵ is H or —(CH₂)_{ng}, X^{c}, wherein ng is 0, 1, 2, 3, 4, 5, or 5, and X^{c} is H, CONH₂, COOH, PO₃H₂, SO₃H, SH, SCH₃, OH, OCH₃, NH₂, NHCH₃, N(CH₃)₂, or N(CH₃)₃.

In certain embodiments, R³ is —(CH₂)ₙₕ, X^{h}, wherein nh is 1, 2, 3, 4 or 5, and X^{h} is H, phenyl, CONH₂, COOH, PO₃H₂, SO₃H, SH, SCH₃, OH, OCH₃, NH₂, NHCH₃, N(CH₃)₂, or N(CH₃)₃. Similarly, in embodiments, R⁴ is —(CH₂)ₙᵢ, Xⁱ, wherein ni is 1, 2, 3, 4 or 5, and Xⁱ is H, phenyl, CONH₂, COOH, PO₃H₂, SO₃H, SH, SCH₃, OH, OCH₃, NH₂, NHCH₃, N(CH₃)₂, or N(CH₃)₃. Preferred groups for R³ and R⁴ include methyl, ethyl, propyl, isopropyl, or n-butyl, —(CH₂)₂COOH, —(CH₂)₃COOH, —(CH₂)₄COOH, —(CH₂)₂SO₃H, —(CH₂)₃SO₃H, —(CH₂)₄SO₃H, —(CH₂)₂PO₃H₂, —(CH₂)₃PO₃H₂, —(CH₂)₄PO₃H₂, —(CH₂)₂N(CH₃)₃, —(CH₂)₃N(CH₃)₃, —(CH₂)₄N(CH₃)₃.

In some preferred embodiments, R³ and R⁴ are the same, for example methyl or ethyl.

In other embodiments, R³ and R⁴ together form a five or six membered ring. Exemplary ring systems include:

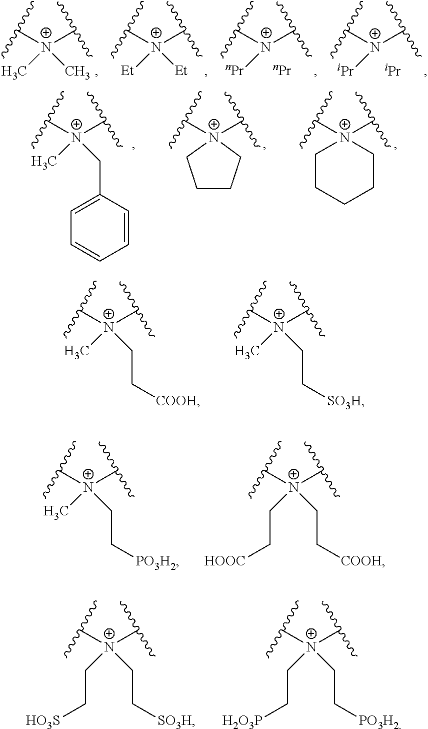

The compounds disclosed herein may be prepared from an oxo-substituted nitrogen heterocycle, e.g., 4-piperidone, first by quaternizing the nitrogen atom, following by double-formylation. In some embodiments, the formylation is conducted using Vilsmeier chemistries:

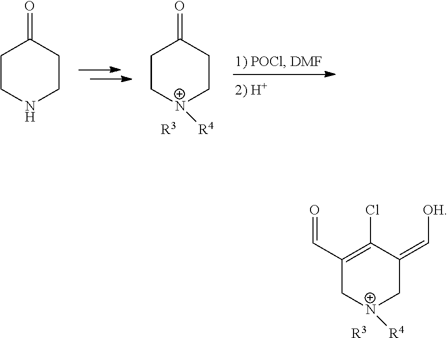

The 4-chloropiperidinum compound may be combined with heterocyclic bases having the formula:

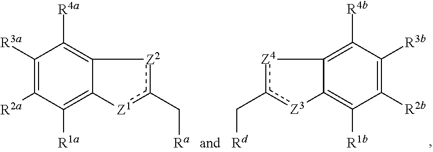

wherein R^{1a}, R^{2a}, R^{3a}, R^{4a}, Z¹, Z², R^{a}, R^{d}, Z³, Z⁴, R^{1b}, R^{2b}, R^{3b}, and R^{4b} have the meanings given above. In preferred embodiments, R^{a} and R^{d} are both hydrogen atoms. The bases may be combined with the 4-chloropiperidinum compound under mildly basic conditions to give the following chlorocyanine compound:

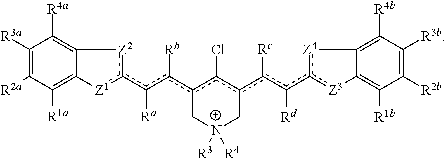

The chlorocyanine may be converted to compounds of the instant invention by reaction with an amine of formula HNR¹R².

The compounds disclosed herein may be formulated with one or more pharmaceutically acceptable carriers and/or excipients in order to be administered to a subject, for example a human. Physiologically acceptable carriers can include water, saline, and may further include agents such as buffers, and other agents such as preservatives that are compatible for use in pharmaceutical formulations. The preferred carrier is a fluid, preferably a liquid, more preferably an aqueous solution; however, carriers for solid formulations, topical formulations, inhaled formulations, ophthalmic formulations, and transdermal formulations are also contemplated as within the scope of the invention. In addition, the pharmaceutical compositions can include one or more stabilizers in a physiologically acceptable carrier. Suitable example of stabilizers for use in such compositions include, for example, low molecular weight carbohydrates, for example a linear polyalcohol, such as sorbitol, and glycerol. Other low molecular weight carbohydrates, such as inositol, may also be used.

It is contemplated that the compounds of the invention can be administered orally or parenterally. For parenteral administration, the compounds can be administered intravenously, intramuscularly, cutaneously, percutaneously, subcutaneously, rectally, nasally, vaginally, and ocularly. Thus, the composition may be in the form of, e.g., solid tablets, capsules, pills, powders including lyophilized powders, colloidal suspensions, microspheres, liposomes granulates, suspensions, emulsions, solutions, gels, including hydrogels, pastes, ointments, creams, plasters, irrigation solutions, drenches, osmotic delivery devices, suppositories, enemas, injectables, implants, sprays, or aerosols. The pharmaceutical compositions can be formulated according to conventional pharmaceutical practice (see, for example, Remington: The Science and Practice of Pharmacy, 20th edition, 2000, ed. A. R. Germaro, Lippincott Williams & Wilkins, Philadelphia, and Encyclopedia of Pharmaceutical Technology, eds. J. Swarbrick and J. C. Boylan, 1988-1999, Marcel Dekker, New York).

The compounds disclosed herein may be used for in vivo imaging. For example, first by administering to a subject a compound disclosed herein, optionally in a pharmaceutical formulation including one or more pharmaceutically acceptable carriers and/or excipients; (b) allowing the compound to distribute within the subject; (c) exposing the subject to light of a wavelength absorbable by the compound and (d) detecting a signal emitted by the agent. Preferably the signal emitted is acoustic (i.e., soundwaves) in nature. Typically, the wavelength of light is in the near infrared range (~650-950 nm or in the near infrared II range (~1,000-1,700 nm). A preferred wavelength is within the water transparency window, e.g., 800-1,400 nm In other embodiments, the subject is exposed to light having a wavelength from 650-1,000 nm, 650-950 nm, 650-900 nm, 650-850 nm, 650-800 nm, 650-750 nm, 650-700 nm, 700-900 nm, 700-800 nm, 750-850 nm, 800-1,000 nm, 850-1,000 nm, 950-1050 nm, 1,000-1,100 nm, 1,050-1,200 nm, 1,100-1,300 nm, 1,200-1,400 nm, or 1,300-1,500 nm.

In some embodiments, the compounds disclosed herein have differing absorption and emittance profiles depending on the local pH environment. Upon protonating (or deprotonation) the electronic and/or spatial configuration of a compound can change, thereby producing a different absorption/emission profile than the compound in the initial state. As such, the compounds disclosed herein may be used to assess the pH in a given system (including biological systems). In some embodiments, the compounds can be used to detect systems, e.g., biological fluids, cells, organs, and/or tissues, having abnormal pH levels.

In some embodiments, a given tissue/cell sample may be determined to be cancerous/diseased if it has a pH less than about 7.2, less than about 7.0, less than or about 6.8, less than or about 6.6, less than about 6.4, less than about 6.2, or less than about 6.0. In some embodiments, a given tissue/cell sample may be determined to be cancerous/diseased if it has a pH from 6.0-7.2, from 6.0-7.0, from 6.0-6.8, from 6.0-6.6, from 6.0-6.4, from 6.0-6.2, from 6.2-7.2, from 6.2-7.0, from 6.2-6.8, from 6.2-6.6, from 6.2-6.4, from 6.4-7.2, from 6.4-7.0, from 6.4-6.8, or from 6.4-6.6. When a system is so determined to be cancerous or otherwise diseased, the subject may been be subjected to one or more modes of therapy to repair, remove, or destroy the cancerous/diseased system. For example, the cancerous/diseased system may be surgically removed, the cancerous/diseased system may be subjected to ionizing radiation, or the subject may be administered one or more chemotherapeutic agents.

In some embodiments, the compounds disclosed herein have differing absorption and emission profiles depending on local concentrations of a given metal ion species. Upon coordination (or de-coordination) of a metal with the compound, the electronic and/or spatial configuration of a compound can change, thereby producing a different absorption/emission profile than the compound in the initial state. As such, the compounds disclosed herein may be used to quantitate detect abnormal metal concentrations in a given system, including biological fluids, cells, organs, and/or tissues.

For example, a system may be contacted with one more or compounds of the invention and irradiated at one or more wavelengths of light. In some embodiments the observed emissions profile at a given site may be used to determine the pH or metal concentration. In certain embodiments, the compounds are characterized by different resonance frequencies at different pH or metal-complexation levels. These differences may be exploited in a variety of different therapeutic contexts. In some embodiments, a system is contacted with a compound of the invention, and then irradiated at a wavelength that matches the resonance frequency of the compound in the protonated and/or metal-complexed state. At least a portion of the absorbed energy is converted to vibrational energy which creates ultrasound waves. These ultrasound waves can be detected, thereby providing real-time differentiation of tissue systems in which the compound is protonated (i.e., lower pH) or metal-complexed than systems in which the compound is not protonated or complexed with a metal. Such methods may be especially advantageously employed in the surgical resections of cancerous cells and tissues. For example, a subject may be treated with one or more compounds of the invention and then irradiation with light have a wavelength matching the resonance frequency of the compound's protonated (or metal complexed) state. The resulting ultrasound waves can be used to differentiate healthy and diseased tissue, permitting the clinician to selectively remove the latter.

In other embodiments, the compounds of the invention may be used to selectively destroy cancerous/diseased tissues by heating and/or ablation. In such embodiments, a subject is administered one or more compounds of the invention and then irradiated with light having a wavelength corresponding to a resonance frequency of the compound in the protonated/metal-complexed state. At least a portion of the induced vibrational energy is converted to heat, which can rupture, destroy, or otherwise inactivate a cell/tissue system wherein the compound is protonated or complexed to metal. Because unprotonated/non-metal complexed compounds do not absorb energy at the relevant wavelength, healthy cells/tissues are not damaged by the method, and the distribution of the compound into healthy cells is not a concern.

In some embodiments, the compounds disclosed herein may be used to identify cells and tissues in a diseased state. In certain embodiments, the disease is selected from the group consisting of bone disease, cancer, cardiovascular disease, a neurogenerative disease, environmental disease, dermatological disease, a bone disease, trauma (e.g., injury), cell death, an autoimmune disease, immunologic disease, inherited disease, infectious disease, inflammatory disease, metabolic disease, and ophthalmic disease. Any cell type, tissue or organ can be monitored including for example, liver, kidney, pancreas, heart, blood, urine, plasma, eyes, CNS (brain), PNS, skin, solid tumors, etc. In preferred embodiments, the disease state is cancer, especially breast cancer, pancreatic cancer, melanoma.

The compounds disclosed herein may be used in the detection of primary tumors, as well as circulating tumor cells and fragments. The compounds may be used to image cancer metathesis through the lymphatic system.

Also disclosed herein is a method for imaging a system (for example a mammal, e.g., a human), including the steps of: (a) adding a compound according to any preceding claim to the system, (b) exposing the system to electromagnetic radiation having wavelength from 680-2,000 nm. After absorbing electromagnetic radiation, the resulting soundwaves may be detected, and optionally converted into an image. In preferred embodiments, the system is irradiated at two or more different wavelengths, and the soundwaves emitted at each wavelength can be detected and compiled. The difference between the first wavelength and second wavelength can be least 5 nm, at least 10 nm, at least 20 nm, at least 30 nm, at least 40 nm, at least 50 nm, at least 60 nm, at least 70 nm, at least 80 nm, at least 90 nm, at least 100 nm, at least 110 nm, at least 120 nm, at least 130 nm, at least 140 nm, or at least 150 nm.

### EXAMPLES

The following examples are for the purpose of illustration of the invention only and are not intended to limit the scope of the present invention in any manner whatsoever.

### Example 1: Synthesis of Quaternary Ammonium Dyes

N-methyl-4-piperidone was first alkylated using methyl iodide to obtain compound 1 as shown below. The final modified Vilsmeier-Haack linker 2 was obtained by following a reported procedure.

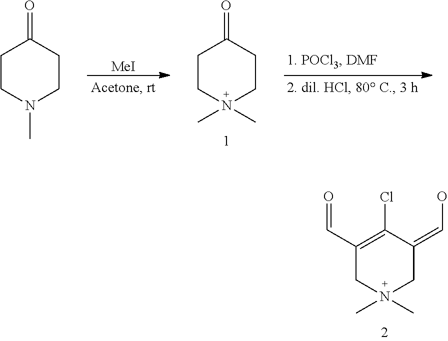

Briefly, anhydrous dimethylformamide was allowed to stir at 0° C. and phosphorus oxychloride was slowly added. The resulting mixture was allowed to stir for about 30 min under cold conditions to generate the Vilsmeier Haack intermediate. Compound 1 was then added in portion to the resulting mixture and the temperature was gradually increased to 80° C. The mixture was stirred continuously at this temperature for about 3 hr. The product obtained was cooled to room temperature and then placed in an ice bath, 1N HCl was added dropwise through an addition funnel whiles stirring to precipitate compound 2 as dark brown solid which was later stored in at 0° C.

The synthesis of the final aminocyanine dyes 52-101 begun first by preparing the heptamethine cyanine dyes (38-51) which contain cyclohexene ring within the polymethine chain. To achieve that, a Fisher indole reaction was used to obtain the heterocyclic salts 26-37. Phenyl hydrazine derivatives bearing electron withdrawing groups such as (chlorine and bromine), and electron donating group (Methoxy) were allowed to react with 3-methyl-2-butanone in acetic acid under reflux condition while stirring vigorously for 24-48 h. N-alkylation of the cyclized intermediate obtained through the Fisher indole step was carried out using six different alkylating agents (methyl iodide, butyl iodide, benzyl bromide, phenylpropyl bromide, 1,4-butanesulftone, and trimethyl propylammonium bromide) in acetonitrile under reflux conditions for 24-48 h. After the reaction, the mixture was allowed to cool to room temperature and the solvents were concentrated under reduced pressure. The resultant products were either poured into ether or ethyl acetate to precipitate the final heterocycle salts 26-37. The yield obtained for the final salts ranged from 70-80%.

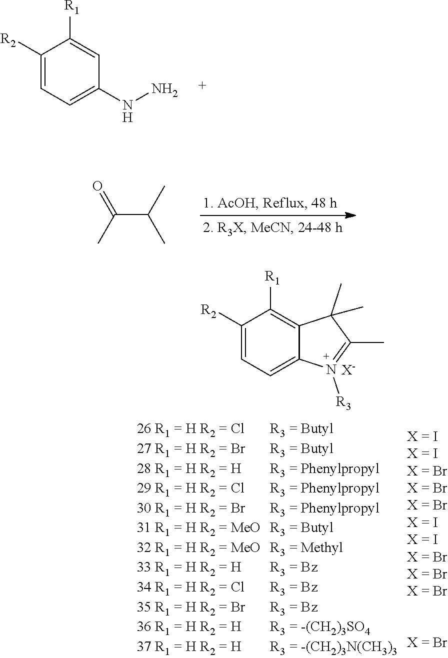

A condensation reaction between the heterocycle salts 26-37 and the linker, compound 25 in acetic anhydride were carried out at an elevated temperature of 80° C. for about 4-6 h. Two equivalents of the heterocycle salts 26-37 synthesized above were allowed to react with one equivalent of compound 25, and three equivalents of sodium acetate. Sufficient amount of acetic anhydride was added to the reaction mixture and the resultant components were heated under reflux for about 4-6 h until total consumption of the starting material, which was indicated by UV-Vis spectrophotometer and thin layer chromatography (TLC). The reaction mixture was allowed to cool to room temperature and either diethyl ether or ethyl acetate was added to precipitate the crude products. In order to obtain the desire compounds 26-37, the crude product obtained were purified using column chromatography with DCM:MeOH (10:1) as the eluting solvent or purified using DMSO:E.A coprecipitation method. The yields of the purified compounds were within 75-80%. The structure of these dyes were characterized and confirmed using ¹H NMR and ¹³C NMR.

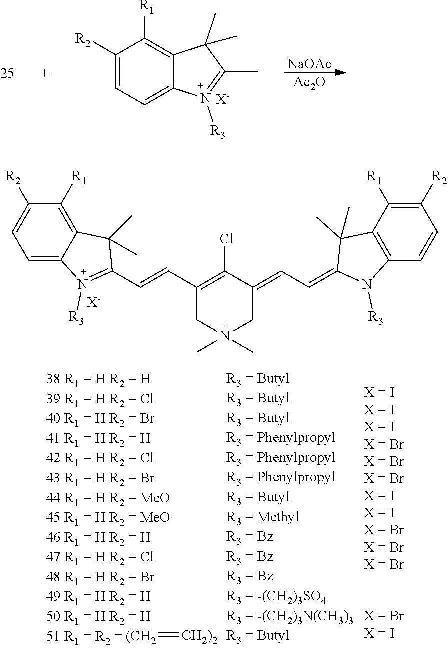

¹H NMR spectrum of compound 38 reveals a triplet signal at 0.99 ppm with coupling constant of 7.28 Hz. This signal corresponds to six protons of the butyl substituents at the nitrogen atom of the heterocyclic ring. A multiplet signal is also observed between 1.55-1.67 ppm which also corresponds to four protons of the butyl group. A very strong intense singlet peak is seen at 1.74 ppm which corresponds to twelve protons of —CH₃ groups attached to the two indolenium rings of the compound. Between 1.82-1.89 ppm another multiplet peak corresponding to four protons of the butyl chain is observed. The two methyl groups attached to the quaternary ammonium nitrogen is observed as a singlet with chemical shift of 3.79 ppm corresponding to six protons. Methylene protons of the butyl chain directly connected to the nitrogen atom of the heterocyclic ring is observed as a triplet at 4.63 ppm with coupling constant of 7.12 Hz. At 5.34 ppm a singlet peak is observed which correspond to the four protons of the connected to the two carbons in the heptamethine linker. Two doublets signals are observed at 6.91 ppm and 8.30 ppm with coupling constant 14.72 Hz. This high coupling constant indicates that the two neighboring protons are trans to each other instead of cis configuration. These two doublets signals observed corresponds to the two protons of the sp² carbons within the polymethine chain. Another two doublet signals are observed at 7.23 ppm and 7.34 ppm with coupling constant of 7.40 Hz. Each of these signals corresponds to two protons of the heterocycle rings. A triplet signal is also observed at 7.42 ppm with coupling constant of 7.25 Hz, which corresponds to four protons of the indolenium rings.

Optoacoustic aminocyanine probes 52-101 were synthesized via S_{N}R1 reaction using different alkylamines (piperazine, N-methyl piperazine, 2-(piperazin-1-yl) ethan-1-ol, piperidin-4-ylmethanol, piperidine-4-carboxylic acid, and thiomorpholine) under anhydrous conditions. Compounds 38-51 were dissolved in anhydrous dimethylformamide under nitrogen conditions whiles stirring. Five to six equivalents of the alkylamines were then added and the resulting mixture were heated under reflux for about 4-24 h.

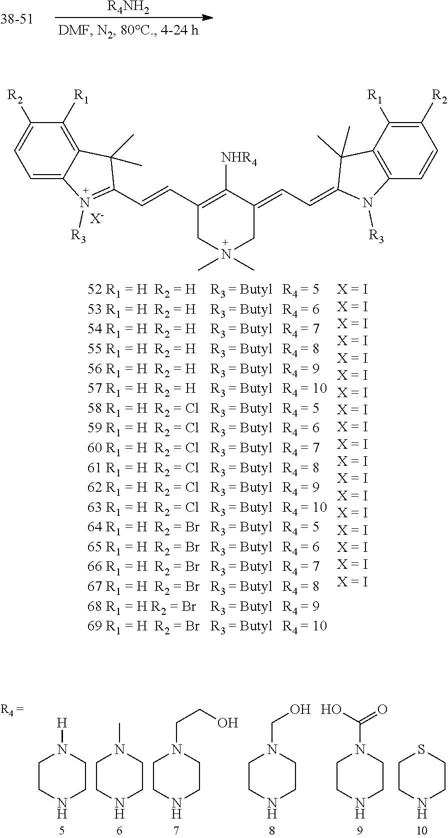

In general, S_{N}R1 reaction between the aminocyanine heptamethine dyes 38-51 and the alkyl amines, (piperazine, N-methyl piperazine, 2-(piperazin-1-yl) ethan-1-ol, piperidin-4-ylmethanol, piperidine-4-carboxylic acid, and thiomorpholine), in anhydrous dimethylformamide under nitrogen conditions were carried out at an elevated temperature of 80° C. for about 4-24 h. One equivalent of the dyes 38-51 synthesized above were allowed to react with five equivalents of alkylamines. The resultant components were heated under reflux until total consumption of the starting material, which was indicated by UV-Vis spectrophotometer and thin layer chromatography (TLC). The reaction mixture was allowed to cool to room temperature and ethyl acetate was added to precipitate the crude products. In order to obtain the desire compounds 52-101, the crude products obtained were purified using column chromatography with DCM:MeOH (10:1) as the eluting solvent or purified using coprecipitation methods using DMSO:E.A. The yields of the purified compounds were within 60-80%.

The following compounds were prepared according to analogous procedures:

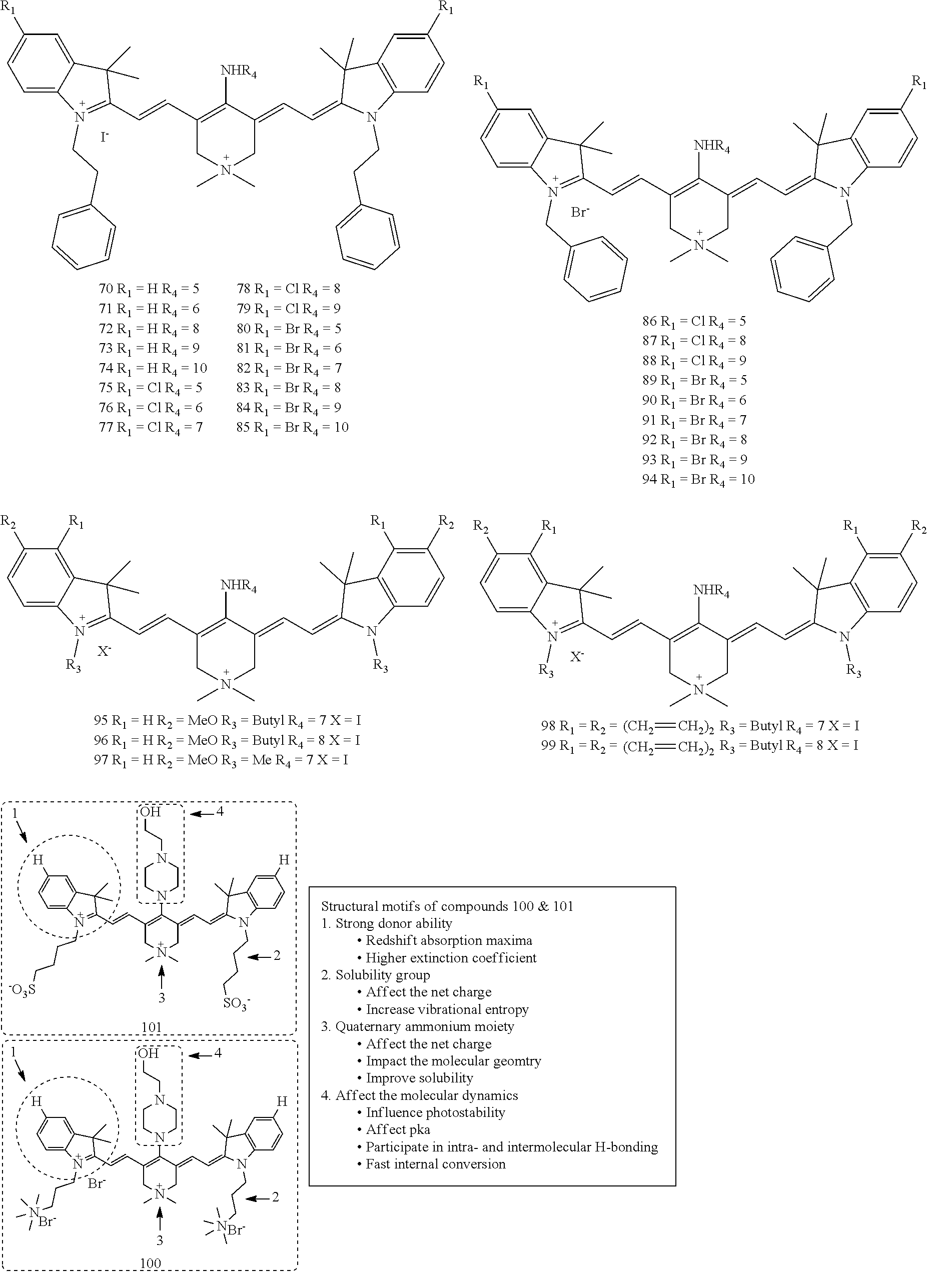

Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-117. (canceled)

118. A compound having the formula: 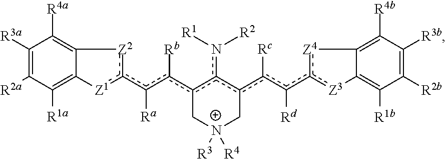
wherein
Z¹ is N—R^{na} and Z² is O, S, C(R^{z2})₂, NR^{na}, N═CR^{z2}, or C(R^{z2})═C(R^{z2}); or Z¹ is null and Z² is Z*—N(R^{na})—, wherein:
Z* is N═CR^{z2}, C(R^{z2})═C(R^{z2}); O, S, or C(R^{z2})₂;
R^{na} is in each case independently selected from C₁₋₈alkyl, optionally substituted one or more times by aryl, heteroaryl, COOH, OH, NH₂, NHCH₃, N(CH₃)₂, N(CH₃)₃, PO₃H₂, SO₃H,
R^{z2} is in case independently selected from F, Cl, Br, I, NO₂, CN, R^{z2*}, OR^{z2*}, N(R^{z2*})₂, SO₃R^{z2}, SO₂R^{z2*}, SO₂N(R^{z2*})₂, C(O)R^{z2*}; C(O)OR^{z2*}, OC(O)R^{z2*}; C(O)N(R^{z2*})₂, N(R^{z2*})C(O)R^{z2}, OC(O)N(R^{z2*})₂, N(R^{z2*})C(O)N(R^{z2*})₂, wherein R^{z2*} is in each case independently selected from hydrogen, C₁₋₈alkyl, aryl, C₁₋₈heteroaryl, C₃₋₈cycloalkyl, or C₁₋₈ heterocyclyl;
Z³ is N—R^{nb} and Z⁴ is O, S, C(R^{z4})₂, NR^{nb}, N═CR^{z4}, or C(R^{z4})═C(R^{z4}); or Z³ is null and Z⁴ is Z*—N(R^{nb})—, wherein:
Z* is N═CR^{z4}, C(R^{z4})═C(R^{z4}), O, S, or C(R^{z4})₂;
R^{nb} is in case independently selected from C₁₋₈alkyl, optionally substituted one or more times by aryl, heteroaryl, COOH, OH, NH₂, NHCH₃, N(CH₃)₂, N(CH₃)₃, PO₃H₂, SO₃H;
R^{z4} is in case independently selected from F, Cl, Br, I, NO₂, CN, R^{z4}, OR^{z4}, N(R^{z4*})₂, SO₃R^{z4}, SO₂R^{z4}, SO₂N(R^{z4*})₂, C(O)R^{z4}; C(O)OR^{z4*}, OC(O)R^{z4*}; C(O)N(R^{z4*})₂, N(R^{z4*})C(O)R^{z4*}, OC(O)N(R^{z4*})₂, N(R^{z4*})C(O)N(R^{z4*})₂, wherein R^{z4*} is in each case independently selected from hydrogen, C₁₋₈alkyl, aryl, C₁₋₈heteroaryl, C₃₋₈cycloalkyl, or C₁₋₈ heterocyclyl;
R^{1a} is F, Cl, Br, I, NO₂, CN, R^{1a*}, OR^{1a*}, SR^{1a*}, N(R^{1a*})₂, SO₃R^{1a*}, SO₂R^{1a*}, SO₂N(R^{1a*})₂, C(O)R^{1a*}; C(O)OR^{1a*}, OC(O) R^{1a*}; C(O)N(R^{1a*})₂, N(R^{1a*})C(O)R^{1a*}, OC(O)N(R^{1a*})₂, N(R^{1a*})C(O)N(R^{1a*})₂, wherein R^{1a*} is in each case independently selected from hydrogen, C₁₋₈alkyl, aryl, C₁₋₈heteroaryl, C₃₋₈cycloalkyl, or C₁₋₈heterocyclyl;
R^{2a} is F, Cl, Br, I, NO₂, CN, R^{2a*}, OR^{2a*}, SR^{2a*}, N(R^{2a*})₂, SO₃R^{2a*}, SO₂R^{2a*}, SO₂N(R^{2a*})₂, C(O)R^{2a*}; C(O)OR^{2a*}, OC(O)R^{2a*}; C(O)N(R^{2a*})₂, N(R^{2a*})C(O)R^{2a*}, OC(O)N(R^{2a*})₂, N(R^{2a*})C(O)N(R^{2a*})₂, wherein R^{2a*} is in each case independently selected from hydrogen, C₁₋₈alkyl, aryl, C₁₋₈heteroaryl, C₃₋₈cycloalkyl, or C₁₋₈heterocyclyl;
R^{3a} is F, Cl, Br, I, NO₂, CN, R^{3a*}, OR^{3a*}, SR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl;
R^{4a} is F, Cl, Br, I, NO₂, CN, R^{4a*}, OR^{4a*}, SR^{4a*}, N(R^{4a*})₂, SO₃R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a*} is in each case independently selected from hydrogen, C₁₋₈alkyl, aryl, C₁₋₈heteroaryl, C₃₋₈cycloalkyl, or C₁₋₈heterocyclyl;
R^{1b} is F, Cl, Br, I, NO₂, CN, R^{1b*}, OR^{1b*}, SR^{1b*}, N(R^{1b*})₂, SO₃R^{1b*}, SO₂R^{1b*}, SO₂N(R^{1b*})₂, C(O)R^{1b}*; C(O)OR^{1b*}, OC(O) R^{1b}*; C(O)N(R^{1b*})₂, N(R^{1b*})C(O)R^{1b*}, OC(O)N(R^{1b*})₂, N(R^{1b*})C(O)N(R^{1b*})₂, wherein R^{1b*} is in each case independently selected from hydrogen, C₁₋₈alkyl, aryl, C₁₋₈heteroaryl, C₃₋₈cycloalkyl, or C₁₋₈heterocyclyl;
R^{2b} is F, Cl, Br, I, NO₂, CN, R^{2b*}, OR^{2b*}, SR^{2b*}, N(R^{2b*})₂, SO₃R^{2b*}, SO₂R^{2b*}, SO₂N(R^{2b*})₂, C(O)R^{2b*}; C(O)OR^{2b*}, OC(O)R^{2b*}; C(O)N(R^{2b*})₂, N(R^{2b*})C(O)R^{2b*}, OC(O)N(R^{2b*})₂, N(R^{2b*})C(O)N(R^{2b*})₂, wherein R^{2b*} is in each case independently selected from hydrogen, C₁₋₈alkyl, aryl, C₁₋₈heteroaryl, C₃₋₈cycloalkyl, or C₁₋₈heterocyclyl;
R^{3b} is F, Cl, Br, I, NO₂, CN, R^{3b*}, OR^{3b*}, SR^{3b*}, N(R^{3b*})₂, SO₃R^{3b*}, SO₂R^{3b*}, SO₂N(R^{3b*})₂, C(O)R^{3b}*; C(O)OR^{3b*}, OC(O)R^{3b*}; C(O)N(R^{3b*})₂, N(R^{3b*})C(O)R^{3b*}, OC(O)N(R^{3b*})₂, N(R^{3b*})C(O)N(R^{3b*})₂, wherein R^{3b*} is in each case independently selected from hydrogen, C₁₋₈alkyl, aryl, C₁₋₈heteroaryl, C₃₋₈cycloalkyl, or C₁₋₈heterocyclyl;
R^{4b} is F, Cl, Br, I, NO₂, CN, R^{4b*}, OR^{4b*}, SR^{4b*}, N(R^{4b*})₂, SO₃R^{4b*}, SO₂R^{4b*}, SO₂N(R^{4b*})₂, C(O)R^{4b}*; C(O)OR^{4b*}, OC(O)R^{4b}*; C(O)N(R^{4b*})₂, N(R^{4b*})C(O)R^{4b*}, OC(O)N(R^{4b*})₂, N(R^{4b*})C(O)N(R^{4b*})₂, wherein R^{4b*} is in each case independently selected from hydrogen, C₁₋₈alkyl, aryl, C₁₋₈heteroaryl, C₃₋₈cycloalkyl, or C₁₋₈heterocyclyl;
R^{a} is F, Cl, Br, I, NO₂, CN, R^{a*}, OR^{a*}, SR^{a*}, N(R^{a*})₂, SO₂R^{a*}, SO₂N(R^{a*})₂, C(O)R^{a*}; C(O)OR^{a*}, OC(O)R^{a*}; C(O)N(R^{a*})₂, N(R^{a*})C(O)R^{a*}, OC(O)N(R^{a*})₂, N(R^{a*})C(O)N(R^{a*})₂, wherein R^{a*} is in each case independently selected from hydrogen, C₁₋₈alkyl, aryl, C₁₋₈heteroaryl, C₃₋₈cycloalkyl, or C₁₋₈heterocyclyl;
R^{b} is F, Cl, Br, I, NO₂, CN, R^{b*}, OR^{b*}, SR^{b*}, N(R^{b*})₂, SO₂R^{b*}, SO₂N(R^{b*})₂, C(O)R^{b*}; C(O)OR^{b*}, OC(O)R^{b*}; C(O)N(R^{b*})₂, N(R^{b*})C(O)R^{b*}, OC(O)N(R^{b*})₂, N(R^{b*})C(O)N(R^{b*})₂, wherein R^{b*} is in each case independently selected from hydrogen, C₁₋₈alkyl, aryl, C₁₋₈heteroaryl, C₃₋₈cycloalkyl, or C₁₋₈heterocyclyl;
R^{c} is F, Cl, Br, I, NO₂, CN, R^{c*}, OR^{c*}, SR^{c*}, N(R^{c*})₂, SO₂R^{c*}, SO₂N(R^{c*})₂, C(O)R^{c*}; C(O)OR^{c*}, OC(O)R^{c*}; C(O)N(R^{c*})₂, N(R^{c*})C(O)R^{c*}, OC(O)N(R^{c*})₂, N(R^{c*})C(O)N(R^{c*})₂, wherein R^{c*} is in each case independently selected from hydrogen, C₁₋₈alkyl, aryl, C₁₋₈heteroaryl, C₃₋₈cycloalkyl, or C₁₋₈heterocyclyl;
R^{d} is F, Cl, Br, I, NO₂, CN, R^{d*}, OR^{d*}, SR^{d*}, N(R^{d*})₂, SO₂R^{d*}, SO₂N(R^{d*})₂, C(O)R^{d*}; C(O)OR^{d*}, OC(O)R^{d*}; C(O)N(R^{d*})₂, N(R^{d*})C(O)R^{d*}, OC(O)N(R^{d*})₂, N(R^{d*})C(O)N(R^{d*})₂, wherein R^{d*} is in each case independently selected from hydrogen, C₁₋₈alkyl, aryl, C₁₋₈heteroaryl, C₃₋₈cycloalkyl, or C₁₋₈heterocyclyl;
R¹ and R² are independently selected from H, C₁₋₆alkyl, C₂₋₆alkenyl, C₂₋₆alkynyl aryl, C₃₋₆ cycloalkyl, C₁₋₆heterocyclyl, or C₁₋₆heteroaryl, wherein R¹ and R² can together form a ring; wherein one or both of R¹ and R² are substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene; with the proviso that when R¹ is H, R² is not phenyl; and
R³ and R⁴ are independently selected from C₁₋₃alkyl, and may together form a ring; wherein each is optionally substituted one or more times by -Q¹-OR^{y}, -Q¹-N(R^{y})₂, -Q¹-N(R^{y′})₃, ═O, aryl, -Q¹-SO₃R^{y}, -Q¹-PO₃(R^{y})₂, -Q¹-C(O)R^{y}, -Q¹-C(O)OR^{y}, -Q¹-C(O)N(R^{y})₂, wherein R^{y} is in each case independently selected from H, C₁₋₆alkyl, R^{y′} is independently selected from C₁₋₃alkyl, wherein any two or more of R^{z} and R^{y′} can together form a ring; and Q¹ is null or C₁₋₄alkylene.

119. The compound of claim 118, wherein R¹ and R² are independently selected from H, C₁₋₆alkyl, C₂₋₆alkenyl, C₂₋₆alkynyl C₃₋₆cycloalkyl, or C₁₋₆heterocyclyl, wherein R¹ and R² can together form a ring; wherein one or both of R¹ and R² are substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆ alkyl, wherein any two or more of R^{z} and R^{z′} can together form a ring; and Q is null or C₁₋₄alkylene.

120. The compound according to claim 119, wherein R¹ and R² are independently selected C₁₋₆alkyl, C₂₋₆alkenyl, and C₂₋₆alkynyl and together form a ring substituted with one or more groups selected from -Q-OR^{z}, -Q-SR^{z}, -Q-N(R^{z})₂, -Q-N(R^{z′})₃, ═O, -Q-CN, C₁₋₆alkyl, aryl, halo, C₁₋₆haloalkyl, C₃₋₆cycloalkyl, C₃₋₆heterocycloalkyl, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆ alkyl, wherein any two or more of R^{z} and R^{z′} can together form a ring; and Q is null or C₁₋₄alkylene.

121. The compound according to claim 118, having the formula:

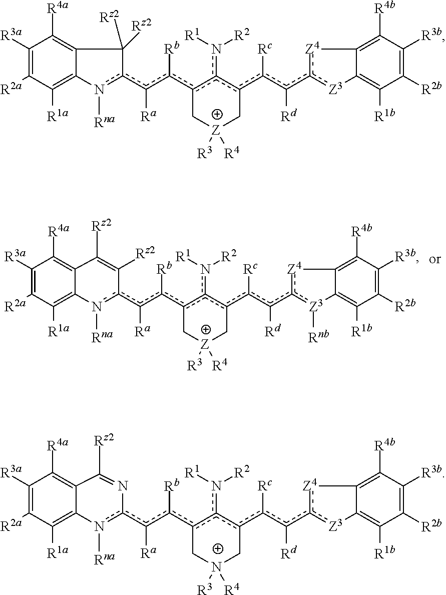

122. The compound of claim 118, having the formula:

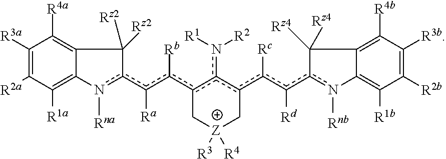

123. The compound according to claim 118, wherein R^{a}, R^{b}, R^{c}, and R^{d} are each hydrogen.

124. The compound according to claim 118, wherein at least one of R^{1a}, R^{2a}, R^{3a}, or R^{4a} are F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene.

125. The compound according to claim 118, wherein at least one of R^{1b}, R^{2b}, R^{3b}, or R^{4b} are F, Cl, Br, I, CN, C₁₋₃alkyl, C₁₋₃haloalkyl, Q¹-OH, Q¹-OC₁₋₃alkyl, Q¹-NH₂, Q¹-NHC₁₋₃alkyl, Q¹-N(C₁₋₃alkyl)₂, Q¹-N⁺(C₁₋₃alkyl)₃, Q¹-SO₃H, Q¹-PO₃H₂, and Q¹-CO₂H, wherein Q¹ is C₁₋₆alkylene.

126. The compound according to claim 122, wherein R^{na} is —(CH₂)ₙₐ—X^{a}, wherein na is 1, 2, 3, 4, 5, 6, 7, or 8, and X^{a} is aryl, heteroaryl, H, COOH, PO₃H₂, SO₃H, OH, NH₂, NHCH₃, N(CH₃)₂, N(CH₃)₃; and wherein R^{nb} is —(CH₂)_{nb}—X^{b}, wherein nb is 1, 2, 3, 4, 5, 6, 7, or 8, and X^{b} is aryl, heteroaryl, H, COOH, PO₃H₂, SO₃H, OH, NH₂, NHCH₃, N(CH₃)₂, N(CH₃)₃.

127. The compound according to claim 118, wherein R^{na} and R^{nb} are the same, and selected from methyl, ethyl, propyl, butyl, benzyl, —(CH₂)₃—N(CH₃)₃, —(CH₂)₃-Ph, and —(CH₂)₃—SO₃H.

128. The compound according to claim 118, wherein R¹ is H and R² is C₁₋₈alkyl, C₃₋₈ cycloalkyl, or C₁₋₈heterocyclyl, substituted with one or more groups selected from -Q-OR^{z}, -Q-SR^{z}, -Q-N(R^{z})₂, -Q-N(R^{z})₃, -Q-SO₂R^{z}, -Q-SO₃R^{z}, -Q-C(O)R^{z}, -Q-C(O)OR^{z}, -Q-C(O)N(R^{z})₂, wherein R^{z} is in each case independently selected from H, or C₁₋₃alkyl, R^{z′} is independently selected from C₁₋₆alkyl, wherein any two or more of R^{z} and R^{z′} can together form a ring; and Q is null or C₁₋₄alkylene.

129. The compound according to claim 118, wherein R¹ and R², together with the nitrogen to which they are attached, form a ring having the formula: 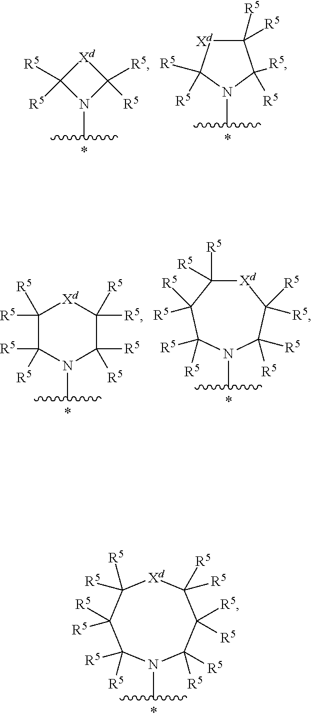
wherein the * represents the bond to the cyanine system,
R⁵ is in each case independently selected from F, Cl, Br, I, NO₂, CN, R^{5a*}, OR^{5a*}, N(R^{5a*})₂, SO₃R^{5a*}, SO₂R^{5a*}, SO₂N(R^{5a*})₂, C(O)R^{5a*}; C(O)OR^{5a*}, OC(O)R^{5a*}; C(O)N(R^{5a*})₂, N(R^{5a*})C(O)R^{5a*}, OC(O)N(R^{5a*})₂, N(R^{5a*})C(O)N(R^{5a*})₂, wherein R^{5a*} is in each case independently selected from hydrogen, C₁₋₈alkyl, aryl, C₁₋₈heteroaryl, C₃₋₈cycloalkyl, or C₁₋₈heterocyclyl;
X^{d} is null, O, S, C(R⁵)₂, or NR⁵,
wherein two geminal R⁵ groups may together form an oxo,
wherein two or more R⁵ groups may together form a ring, and
wherein two adjacent R⁵ groups may together form a double bond.

130. The compound according to claim 118, wherein R¹ and R², together with the nitrogen to which they are attached, form a ring having the formula: 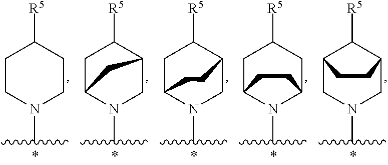
wherein R⁵ is H or —(CH₂)_{nf}, X^{c}, wherein nf is 1, 2, 3, 4, 5, or 5, and X^{c} is H, CONH₂, COOH, PO₃H₂, SO₃H, SH, SCH₃, OH, OCH₃, NH₂, NHCH₃, N(CH₃)₂, or N(CH₃)₃.

131. The compound according to claim 118, wherein R³ is —(CH₂)ₙₕ, X^{h}, wherein nh is 1, 2, 3, 4 or 5, and X^{h} is H, phenyl, CONH₂, COOH, PO₃H₂, SO₃H, SH, SCH₃, OH, OCH₃, NH₂, NHCH₃, N(CH₃)₂, or N(CH₃)₃; and R⁴ is —(CH₂)ₙᵢ, Xⁱ, wherein ni is 1, 2, 3, 4 or 5, and Xⁱ is H, phenyl, CONH₂, COOH, PO₃H₂, SO₃H, SH, SCH₃, OH, OCH₃, NH₂, NHCH₃, N(CH₃)₂, or N(CH₃)₃.

132. The compound according to claim 118, wherein R³ and R⁴ are independently selected from methyl, ethyl, propyl, isopropyl, or n-butyl.

133. The compound according to claim 118, wherein R³ and R⁴ together form a five or six membered ring.

134. The compound of claim 118, wherein R³ and R⁴ together with the nitrogen to which they are attached form a group having the formula:

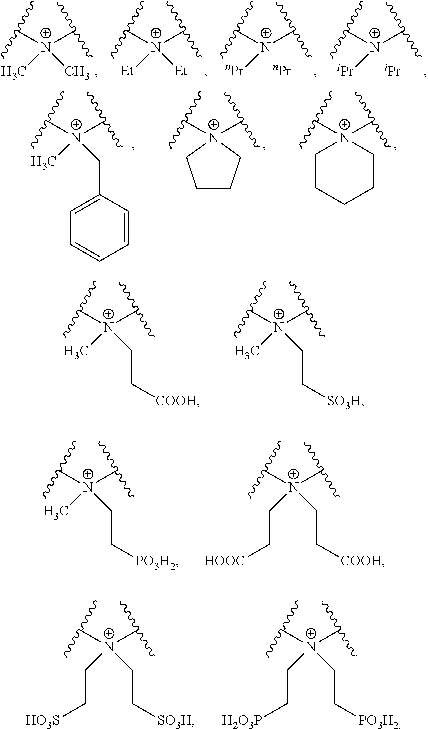

135. A method for imaging a system, comprising adding a compound according to claim 118 to the system, exposing the system to electromagnetic radiation having wavelength from 680-1200 nm.

136. The method of claim 135, further comprising detecting soundwaves produced by the compound subsequent to exposure to electromagnetic radiation.

137. The method of claim 136, comprising exposing the system to multiple wavelengths of electromagnetic radiation, each wavelength producing a different soundwave, and the imaging comprises comparing soundwaves produced at a first wavelength of electromagnetic radiation with soundwaves produced a second wavelength of electromagnetic radiation, said first wavelength being different than the second wavelength.
